(19) United States
(12) Patent Application Publication
(10) Pub. No.: US 2026/0061202 A1
(43) Pub. Date: Mar. 5, 2026

(54) Hybrid Magnetic-Ultrasonic Wireless Interrogation of Millimeter-Scale Biomedical Implants With Magnetoelectric Transducer

(71) Applicant: The Penn State Research Foundation, University Park, PA (US)

(72) Inventors: Sumanta Kumar Karan, University Park, PA (US); Sujay Hosur, State College, PA (US); Bed Poudel, University Park, PA (US); Mehdi Kiani, State College, PA (US); Shashank Priya, University Park, PA (US); Zeinab Kashani, University Park, PA (US)

(73) Assignee: The Penn State Research Foundation, Univeristy Park, PA (US)

(21) Appl. No.: 19/385,766

(22) Filed: Nov. 11, 2025

Related U.S. Application Data
(63) Continuation-in-part of application No. PCT/US2024/058639, filed on Dec. 5, 2024.
(60) Provisional application No. 63/609,482, filed on Dec. 13, 2023.
(60) Provisional application No. 63/721,096, filed on Nov. 15, 2024.

(51) Int. Cl. A61N 1/372; H02J 50/10; H03K 17/687

(52) U.S. Cl. CPC A61N 1/37223; H02J 50/10; H03K 17/6872

(57) ABSTRACT

Embodiments can relate to an implant stimulation and recording system. The system can include a receiver. The system can include an interrogator having a transmitter coil and an ultrasound transducer. The system can include an implant having an integrated circuit and a magnetoelectric transducer. Low-frequency magnetic fields are generated by the transmitter coil and are converted into an electric voltage by the magnetoelectric transducer. Ultrasound energy is generated by the transducer and converted into an electric voltage by the magnetoelectric transducer. When voltage is applied to the magnetoelectric transducer, the magnetoelectric transducer generates magnetic fields and ultrasound waves, wherein the receiver detects the magnetic fields and ultrasound waves generated by the magnetoelectric transducer.

### CROSS-REFERENCE TO RELATED APPLICATIONS

This patent application is a continuation in part application of U.S. provisional application no. 63/721,096, filed on Nov. 15, 2024, the entire contents of which is incorporated herein by reference. This patent application also claims the benefit of priority of PCT/US24/58639, filed on Dec. 5, 2024, the entire contents of which is incorporated by reference.

### STATEMENT REGARDING FEDRALLY SPONSORED RESEARCH OR DEVELOPMENT

This invention was made with government support under Grant No. ECCS1904811 awarded by the National Science Foundation. The government has certain rights in the invention.

### FIELD OF THE INVENTION

Embodiments can relate to implant stimulation and recording system configured for wireless power reception through one modality (magnetic field or ultrasound) and simultaneous uplink data transmission using the other.

### BACKGROUND OF THE INVENTION

Conventional technologies for wireless power transfer in implantable medical devices include inductive, ultrasound (US), and magnetoelectric (ME) modalities. Yet, these conventional systems have to operate at high frequencies (100s of MHz) which suffer from limited external power transfer due to IEEE safety restrictions and high tissue absorption.

### SUMMARY OF THE INVENTION

Additional understanding of the present disclosure can be appreciated from U.S. provisional application No. 63/609,482, filed on Dec. 13, 2023 the entire contents of each being incorporated herein by reference.

Embodiments can relate to an implant stimulation and recording system. The system can include a receiver. The system can include an interrogator having a transmitter coil and an ultrasound transducer. The system can include an implant having an integrated circuit and a magnetoelectric transducer. Low-frequency magnetic fields are generated by the transmitter coil and is converted into an electric voltage by the magnetoelectric transducer. Ultrasound energy is generated by the transducer and converted into an electric voltage by the magnetoelectric transducer. When voltage is applied to the magnetoelectric transducer, the magnetoelectric transducer generates magnetic fields and ultrasound waves, wherein the receiver detects the magnetic fields and ultrasound waves generated by the magnetoelectric transducer.

Further features, aspects, objects, advantages, and possible applications of the present invention will become apparent from a study of the exemplary embodiments and examples described below, in combination with the Figures, and the appended claims.

### BRIEF DESCRIPTION OF THE DRAWINGS

The above and other objects, aspects, features, advantages and possible applications of the present innovation will be more apparent from the following more particular description thereof, presented in conjunction with the following drawings. Like reference numbers used in the drawings may identify like components.

FIG. 1 shows an exemplary MagSonic interrogation method representation with a single magnetoelectric (ME) transducer and an image of an exemplary fabricated ME transducer.

FIG. 2 shows a block diagram of an exemplary completed wireless system for MagSonic operation including the key blocks of an exemplary MagSonic ASIC.

FIG. 3 shows an exemplary integrated power management and data Tx (IPMDT) circuit schematic, with switch S₁ employing a adaptive body bias (ABB) technique, the stacked NMOS transistors acting as S₄ switch, S₂ PMOS switch, and S₃ NMOS switch. FIG. 3 also shows a table indicating the operation of each switch under different operation modes.

FIG. 4 shows circuits schematics of exemplary (a) charge pump and (b) level shifter.

FIG. 5 shows a schematic of a high voltage switch in an exemplary stimulator to ensure reliable operation at voltages up to 6.6 V (2×V_{DD}).

FIG. 6 shows an exemplary AC stimulator architecture with active charge balancing scheme, and a notch detection circuitry for initiating stimulation in each phase.

FIG. 7 shows an exemplary operational flowchart of an exemplary stimulator block with active and passive charge balancing.

FIG. 8 shows an exemplary circuit schematic of an exemplary neural amplifier with variable gain as well as the transistor-level circuits of the op-amps in each stage.

FIG. 9 shows an exemplary implementation of an exemplary the analog-to-time converter. Descriptive waveforms indicating the operation of this circuit and the generation of two data pulses for reliable uplink communication are demonstrated.

FIG. 10 shows measured impedance profiles of an exemplary ME transducer at the (a) length, (b) width, and (c) thickness mode resonance frequencies. All measurements were performed in a water medium.

FIG. 11 shows measured received power (P_{L}) variation of an exemplary ME across load resistances (R_{L}) operating under safety limits in a water medium.

FIG. 12 shows a die micrograph of an exemplary MagSonic ASIC with key blocks labeled.

FIG. 13 shows (a) measured transient waveforms of the power management (voltage doubler with self-regulation) in steady state, (b) measured self-start transient waveforms of the exemplary MagSonic ASIC, and (c) measured power conversion efficiency of the voltage doubler across different load resistances (R_{L}).

FIG. 14 shows (a) measured frequency response of an exemplary neural amplifier for different gain settings, transient waveforms for (b) code 00, and (c) code 01 gain settings with sinusoidal inputs while the exemplary MagSonic ASIC was powered wirelessly.

FIG. 15 shows (a) measured waveforms showing an exemplary AC stimulator operation generating a biphasic output controlled by notches in the power carrier, (b) measured residual voltage vs. number of stimulation pulses, and (c) measured waveforms for resetting an exemplary neural amplifier during the stimulation phase.

FIG. 16 shows stimulation with (a) only MF power and (b) both MF and US powering of an exemplary ME transducer resulting in higher stimulation voltage.

FIG. 17 shows an exemplary MagSonic interrogation demonstration with ASIC including (a) simultaneous US powering with MF uplink data transmission and (b) MF powering with US uplink data transmission.

FIG. 18 shows an exemplary MagSonic link demonstration with ASIC using synthetic neural signal (original and reconstructed waveforms with BER<10⁻³).

FIG. 19 shows (a) fabrication procedure of the flexible electrode used in the animal experiment, (b) image of the fabricated stimulating electrode, (c) measured electrochemical impedance spectroscopy of the fabricated electrode with comparison to a Pt wire, and (d) cyclic voltammetry (CV) measurements of the stimulating electrode and Pt wire.

FIG. 20 shows (a) image of the surgical implantation of the stimulation and reference electrodes inserted into the left hindlimb of the mouse, (b) image captures show the physical movement of the hindlimb without and with stimulation applied, (c) mouse hindlimb deflection for a continuous AC type stimulation applied once every 2 seconds with an exemplary MagSonic ASIC.

FIG. 21 benchmarks an exemplary MagSonic ASIC with state-of-the-art ASICs that utilize wireless interrogation for neural stimulation applications where the implant size is in the mm scale.

FIG. 22 shows an exemplary implant stimulation and recording system.

FIG. 23 shows an exemplary IPMDT circuit.

FIG. 24 shows an exemplary biphasic alternating current stimulator.

FIG. 25 shows an exemplary charge pump circuit.

FIG. 26 shows an exemplary level shifter circuit.

FIG. 27 shows an exemplary high-voltage tolerant switch circuit.

FIG. 28 shows an exemplary biphasic alternating current stimulator.

FIG. 29 shows an exemplary recording low-noise amplifier circuit.

### DETAILED DESCRIPTION OF THE INVENTION

The following description is of exemplary embodiments that are presently contemplated for carrying out the present invention. This description is not to be taken in a limiting sense, but is made merely for the purpose of describing the general principles and features of the present invention. The scope of the present invention is not limited by this description.

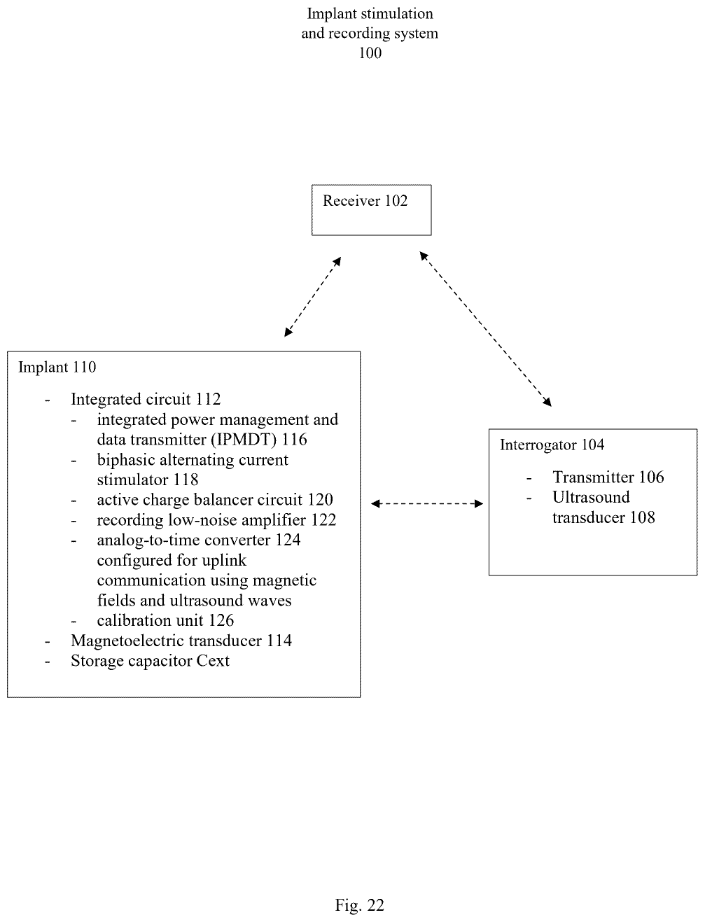

Referring to FIG. 22, an exemplary embodiment can relate to an implant stimulation and recording system 100. The system can include a receiver 102 (e.g., a data receiver circuit). The system 100 can include an interrogator 104. The interrogator 104 can include a transmitter 106 (e.g., a transmitter circuit, transmitter coil, etc.) and an ultrasound transducer 108. The system 100 can include an implant 110 (e.g., a device that tis implantable into an animal, such as an implantable medical device) having an integrated circuit 112 (a collection of electrical circuits on a semiconductor wafer, the circuits including transistors, resistors, capacitors, etc.), a magnetoelectric transducer 114 (e.g., a device configured to convert magnetic energy into electrical energy by coupling a magnetostrictive material that deforms in response to a magnetic field and a piezoelectric material that generates an electrical charge from mechanical stress), and a storage capacitor Cext. In operation, low-frequency magnetic fields are generated by the transmitter 106 and converted into an electric voltage by the magnetoelectric transducer 114. Ultrasound energy is generated by the transducer 114 and converted into an electric voltage by the magnetoelectric transducer 114. When voltage is applied to the magnetoelectric transducer 114, the magnetoelectric transducer 114 generates magnetic fields and ultrasound waves, and the receiver 102 detects the magnetic fields and ultrasound waves generated by the magnetoelectric transducer 114.

The integrated circuit 112 can include an integrated power management and data transmitter (IPMDT) 116, a biphasic alternating current stimulator 118, an active charge balancer circuit 120, a recording low-noise amplifier 112, an analog-to-time converter 124 configured for uplink communication using magnetic fields and ultrasound waves, and a calibration unit 124.

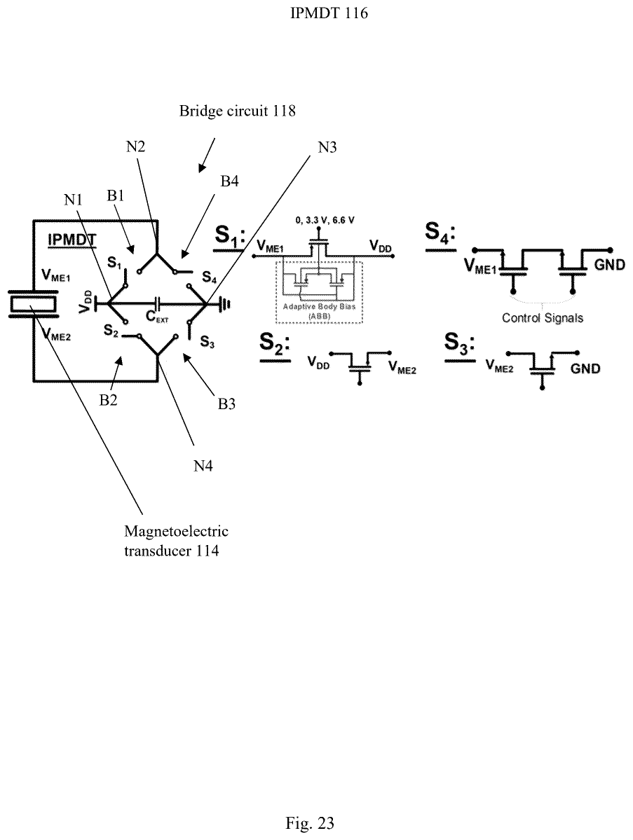

FIG. 23 shows a non-limiting exemplary embodiment of the IPMDT 116. In this example, the IPMDT 116 is connected to a first voltage output VME1 of the magnetoelectric transducer 114 and a voltage output VME2 of the magnetoelectric transducer 114. The IPMDT 116 includes a bridge circuit 118. The bridge circuit 118 includes a first branch B1 with a first switch S1, a second branch B2 with a second switch S2, a third branch B3 with a third switch S3, a fourth branch B4 with a fourth switch S4, and a storage capacitor Cext. B1 is connected to node N1 and node N2. B2 is connected to node N1 and node N4. B3 is connected to node N3 and node N4. B4 is connected to node N3 and node N2. N1 is connected to a positive power supply VDD and to Cext. N3 is connected to ground GND and to Cext. S1 is a P-channel Metal-Oxide-Semiconductor (PMOS) transistor having a source connected to VME1, a gate, and a drain connected to VDD. S2 is a PMOS transistor having a source connected to VME2, a gate, and a drain connected to VDD. S3 is a N-channel Metal-Oxide-Semiconductor (NMOS) transistor having a source connected to VME2, a gate, and a drain connected to GND. S4 includes a first NMOS transistor and a second NMOS transistor. The first NMOS transistor has a source connected to VME1, a gate, and a drain connected to a source of the second NMOS transistor. The second NMOS transistor has the source connected to the drain of the first NMOS transistor, a gate, and a drain connected to GND.

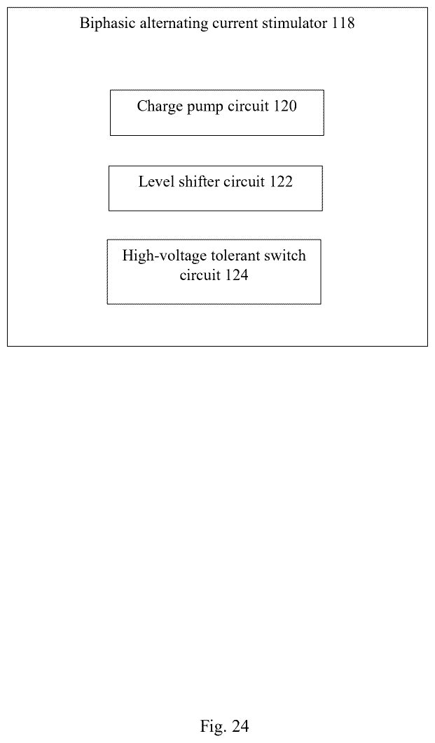

FIG. 24 shows a non-limiting exemplary embodiment of the biphasic alternating current stimulator 118. In this exemplary embodiment, the biphasic alternating current stimulator 118 includes a charge pump circuit 120, a level shifter circuit 122, and a high-voltage tolerant switch circuit 124.

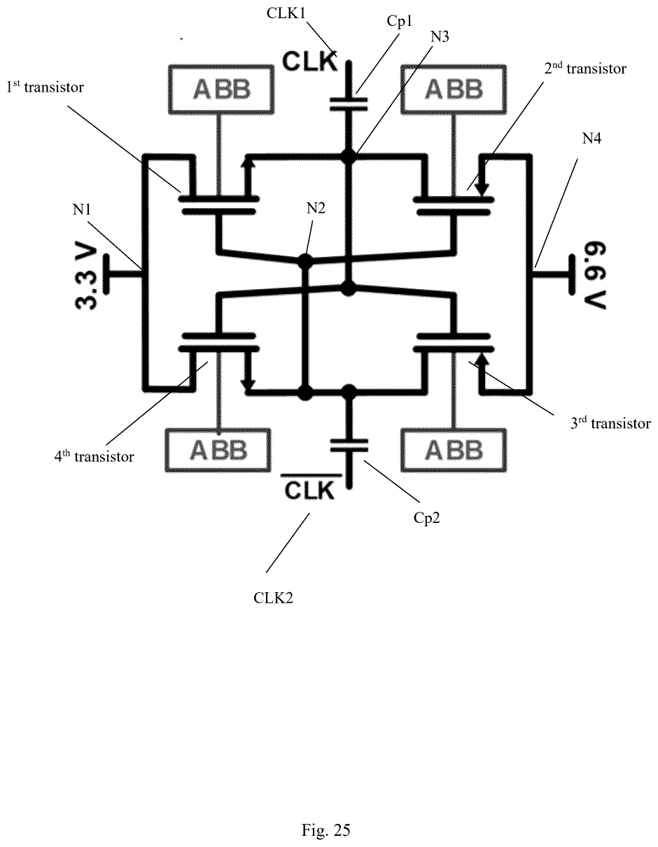

Referring to FIG. 25, the charge pump circuit 120 includes a first NMOS transistor, a second PMOS transistor, a third PMOS transistor, and a fourth NMOS transistor. The first NMOS transistor includes a source connected to node N3, a gate connected to node N2, a drain connected to node N1. The second PMOS transistor includes a source connected to node N4, a gate connected to node N2, a drain connected to node N3. N3 is connected to a pumping capacitor Cp1 whose other end is connected to a first clock CLK1. The third PMOS transistor includes a source connected to node N4, a gate connected to node N3, a drain connected to node N2. A second pumping capacitor Cp2 is connected between a clock CLK2 and node N2. The fourth NMOS transistor includes a source connected to N2, a gate connected to N3, and a drain connected to N1.

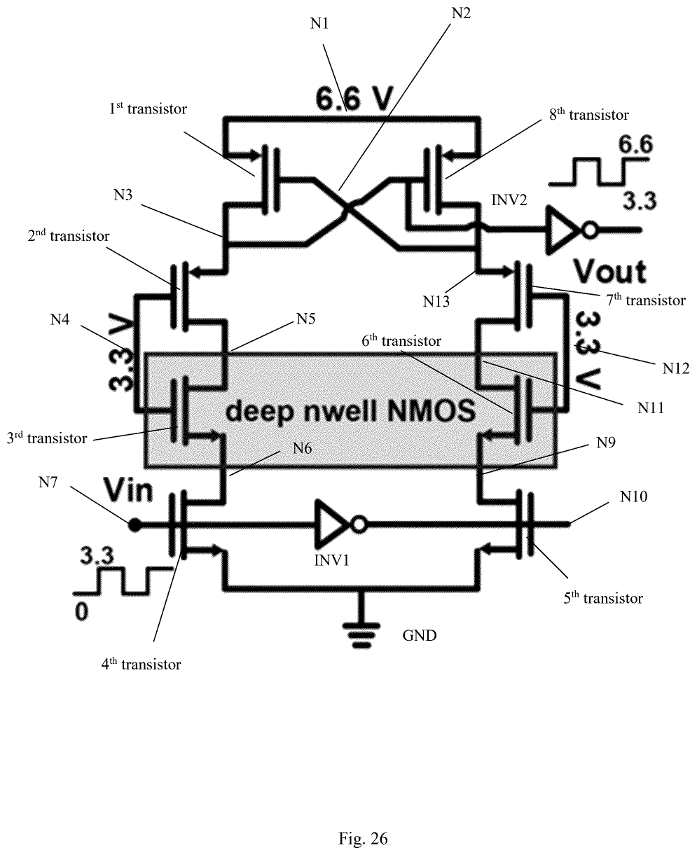

Referring to FIG. 26, the level shifter circuit 122 includes a first PMOS transistor, a second PMOS transistor, a third NMOS transistor, and a fourth NMOS transistor, a fifth NMOS transistor, a sixth NMOS transistor, a seventh PMOS transistor, and an eight PMOS transistor. The first PMOS transistor includes a source connected to node N1, a gate connected to node N2, and a drain connected to node N3. The second PMOS transistor includes a source connected to N3, a gate connected to node N4, and a drain connected to node N5. The third NMOS transistor includes a source connected to N5, a gate connected to N4, and a drain connected to N6. The fourth NMOS transistor includes a source connected to N6, a gate connected to node N7, and a drain connected to GND. N7 is connected to a voltage source (Vin). The fifth NMOS transistor includes a source connected to node N9, a gate connected to node N10, and a drain connected to node GND. N7 is connected to an inverter INV1. The sixth NMOS transistor includes a source connected to node N11, a gate connected to node N12, and a drain connected to N9. The seventh PMOS transistor includes a source connected to node N13, a gate connected to node N12, and a drain connected to N11. The eighth PMOS transistor includes a source connected to N1, a gate connected to N3 and to an inverter INV2, and a drain connected to N13.

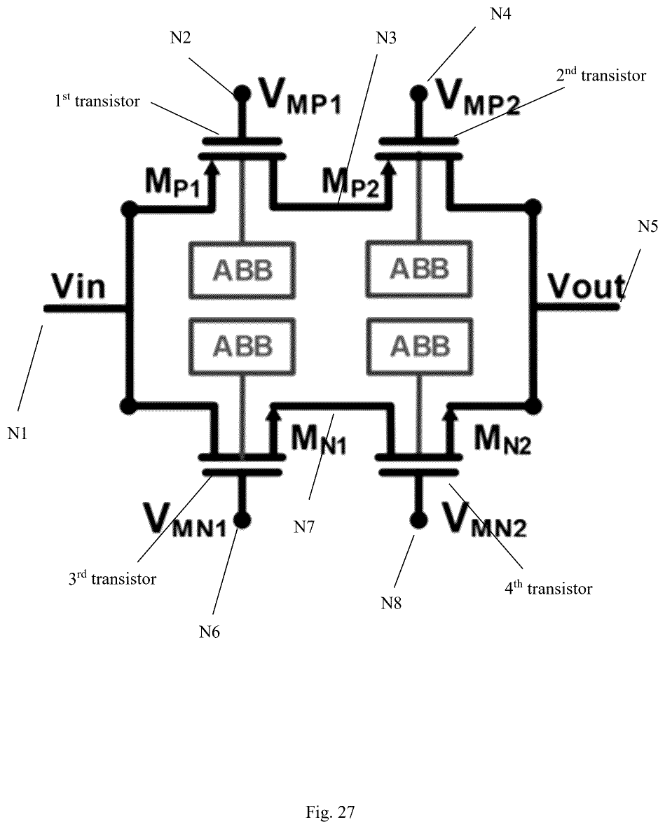

Referring to FIG. 27, the high-voltage tolerant switch circuit 124 includes a first PMOS transistor, a second PMOS transistor, a third NMOS transistor, and a fourth NMOS transistor. The first PMOS transistor includes a source connected to node N1, a gate connected to node N2, and a drain connected to node N3. N2 is connected to a voltage control signal VMP1. The second PMOS transistor includes a source connected to N3, a gate connected to node N4, and a drain connected to node N5. N4 is connected to voltage control signal VMP2. The third NMOS transistor includes a source connected to N7, a gate connected to node N6, and a drain connected to node N1. N6 is connected to voltage control signal VMN1. The fourth NMOS transistor includes a source connected to N5, a gate connected to node N8, and a drain connected to N7. N8 is connected to voltage control signal VMN2.

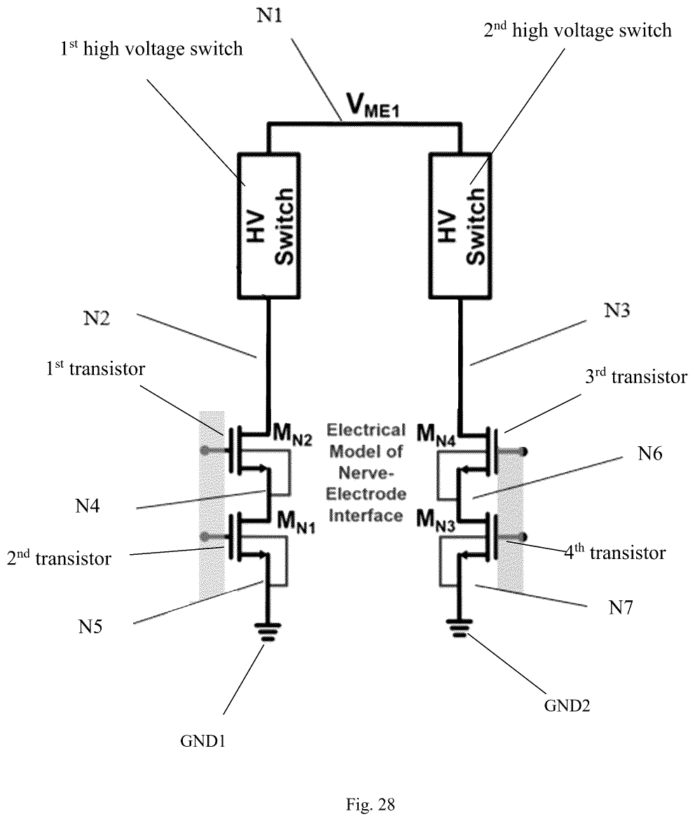

Referring to FIG. 28, the biphasic alternating current stimulator 118 includes a first high voltage switch connected to node N1 and node N2, a second high voltage switch connected to N1 and node N3. N1 is connected to a voltage source VME1. The active biphasic alternating current stimulator 118 includes a first NMOS transistor, a second NMOS transistor, a third NMOS transistor, and a fourth NMOS transistor. The first NMOS transistor includes a drain connected to N2, a gate, and a source connected to node N6. The second NMOS transistor includes a drain connected to N4, a gate, and a source connected to node N5. N5 is connected to a first ground GND1. The third NMOS transistor includes a drain connected to N3, a gate, and a source connected to node N6. The fourth NMOS transistor includes a drain connected to N6, a gate, and a source connected to node N7. N7 is connected to a second ground GND2.

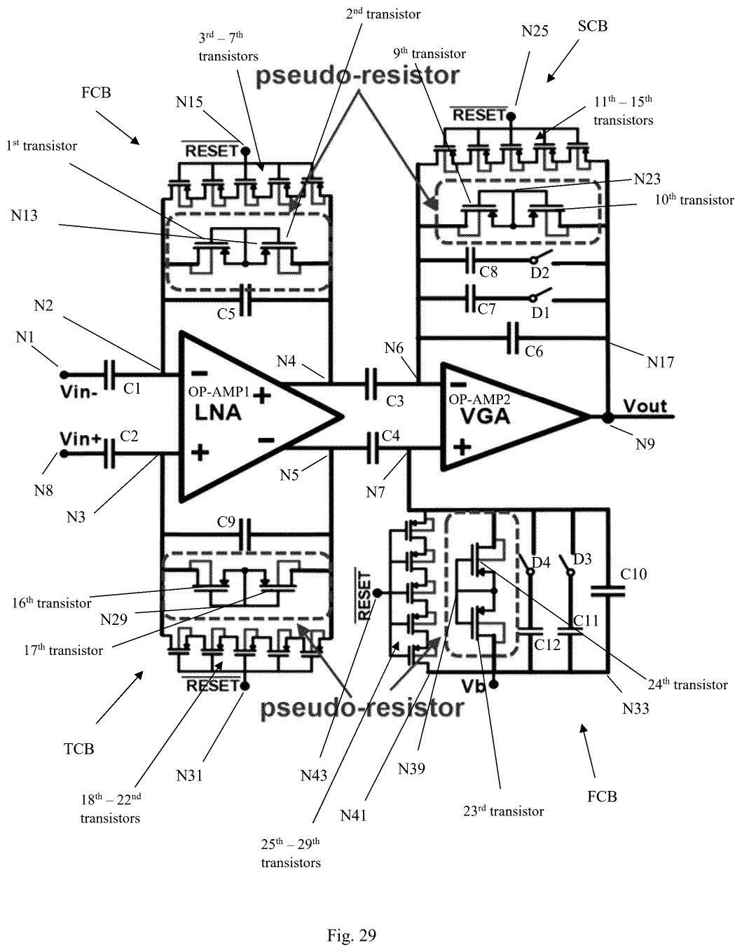

Referring to FIG. 29, the recording low-noise amplifier 122 includes a first op-amp OP-AMP1, a second op-amp OP-AMP2, a first circuit block FCB, a second circuit block SCB, a third circuit block TCB, and a fourth circuit block FCB. OP-AMP1 includes a first input connected to node N2, a second input connected to node N3, a first output connected to node N4, and a second output connected to node N5. N2 is connected to a first capacitor C1, and C1 is connected to node N1. N1 is connected to a voltage source Vin−. N3 is connected to a second capacitor C2, and C2 is connected to node N8. N8 is connected to a voltage source Vin+. N4 is connected to a third capacitor C3, and C3 is connected to node N6. N5 is connected to a fourth capacitor C4, and C4 is connected to node N7. OP-AMP2 includes a first input connected to N6, a second input connected to N7, and an output connected to node N9.

The first circuit block FCB includes a fifth capacitor C5 connected to node N2 and node N4. The first circuit block FCB also includes a first PMOS transistor, a second PMOS transistor, a third PMOS transistor, a fourth PMOS transistor, a fifth PMOS transistor, a sixth PMOS transistor, and a seventh PMOS transistor. The first PMOS transistor includes a drain connected to node N2, a gate connected to node N13, and a source connected to N13. The second PMOS transistor includes a drain connected to node N4, a gate connected to N13, and a source connected to N13. Each of the third, fourth, fifth, sixth, and seventh PMOS transistors includes a source, a gate, and a drain. For the third PMOS transistor, the drain is connected to N2, the gate is connected to node N15, and the source connected to the drain of the fourth PMOS transistor. For the fourth PMOS transistor, the drain is connected to the source of the third PMOS transistor, the gate is connected to node N15, and the source connected to the drain of the fifth PMOS transistor. For the fifth PMOS transistor, the drain is connected to the source of the fourth PMOS transistor, the gate is connected to node N15, and the source connected to the drain of the sixth PMOS transistor. For the sixth PMOS transistor, the drain is connected to the source of the fifth PMOS transistor, the gate is connected to node N15, and the source connected to the drain of the seventh PMOS transistor. For the seventh PMOS transistor, the drain is connected to the source of the sixth PMOS transistor, the gate is connected to node N15, and the source connected to N4.

The second circuit block SCB includes a sixth capacitor C6 connected to node N6 and node N17. N17 is connected to N9. A seventh capacitor C7 is connected to node N6 and node N17 via a first switch D1. N19 is connected to N17. An eighth capacitor C8 is connected to node N6 and node N17 via a second switch D2. The second circuit block SCB includes a ninth PMOS transistor, a tenth PMOS transistor, an eleventh PMOS transistor, a twelfth PMOS transistor, a thirteenth PMOS transistor, a fourteenth PMOS transistor, and a fifteenth PMOS transistor. The ninth PMOS transistor includes a drain connected to node N6, a gate connected to node N23, and a source connected to N23. The tenth PMOS transistor includes a drain connected to node N17, a gate connected to N23, and a source connected to N23. Each of the eleventh, twelfth, thirteenth, fourteenth, and fifteenth PMOS transistors includes a source, a gate, and a drain. For the eleventh PMOS transistor, the drain is connected to N6, the gate is connected to node N25, and the source connected to the drain of the twelfth PMOS transistor. For the twelfth PMOS transistor, the drain is connected to the source of the eleventh PMOS transistor, the gate is connected to node N25, and the source connected to the drain of the thirteenth PMOS transistor. For the thirteenth PMOS transistor, the drain is connected to the source of the twelfth PMOS transistor, the gate is connected to node N25, and the source connected to the drain of the fourteenth PMOS transistor. For the fourteenth PMOS transistor, the drain is connected to the source of the thirteenth PMOS transistor, the gate is connected to node N25, and the source connected to the drain of the fifteenth PMOS transistor. For the fifteenth PMOS transistor, the drain is connected to the source of the fourteenth PMOS transistor, the gate is connected to node N25, and the source connected to N17.

The third circuit block TCB includes a ninth capacitor C9 connected to node N3 and node N5. The third circuit block TCB includes a sixteenth PMOS transistor, a seventeenth PMOS transistor, an eighteenth PMOS transistor, a nineteenth PMOS transistor, a twentieth PMOS transistor, a twenty first PMOS transistor, and a twenty second PMOS transistor. The sixteenth PMOS transistor includes a source connected to node N3, a gate connected to node N29, and a drain connected to N29. The seventeenth PMOS transistor includes a source connected to node N5, a gate connected to N29, and a drain connected to N29. Each of the eighteenth, nineteenth, twentieth, twenty first, and twenty second PMOS transistors includes a source, a gate, and a drain. For the eighteenth PMOS transistor, the drain is connected to N3, the gate is connected to node N31, and the source connected to the drain of the nineteenth PMOS transistor. For the nineteenth PMOS transistor, the drain is connected to the source of the eighteenth PMOS transistor, the gate is connected to node N31, and the source connected to the drain of the twentieth PMOS transistor. For the twentieth PMOS transistor, the drain is connected to the source of the nineteenth PMOS transistor, the gate is connected to node N31, and the source connected to the drain of the twenty first PMOS transistor. For the twenty first PMOS transistor, the drain is connected to the source of the twentieth PMOS transistor, the gate is connected to node N31, and the source connected to the drain of the twenty second PMOS transistor. For the twenty second PMOS transistor, the drain is connected to the source of the twenty first PMOS transistor, the gate is connected to node N31, and the source connected to N5.

The fourth circuit block FCB includes a tenth capacitor C10 connected to node N7 and node N33. The fourth circuit block FCB includes an eleventh capacitor C11 connected to node N7 and node N33 via a third switch D3. The fourth circuit block FCB includes a twelfth capacitor C12 connected to node N7 and node N33 via a fourth switch D4. The fourth circuit block FCB includes a twenty third PMOS transistor, a twenty fourth PMOS transistor, a twenty fifth PMOS transistor, a twenty sixth PMOS transistor, a twenty seventh PMOS transistor, a twenty eighth PMOS transistor, and a twenty ninth PMOS transistor. The twenty third PMOS transistor includes a drain connected to node N33, a gate connected to node N39, and a source connected to N39. The twenty fourth PMOS transistor includes a drain connected to node N7, a gate connected to N39, and a source connected to N39. Each of the twenty fifth, twenty sixth, twenty seventh, twenty eighth, and twenty ninth PMOS transistors includes a source, a gate, and a drain. For the twenty fifth PMOS transistor, the drain is connected to node N41, the gate is connected to node N43, and the source connected to the drain of the twenty sixth PMOS transistor. For the twenty sixth PMOS transistor, the drain is connected to the source of the twenty fifth PMOS transistor, the gate is connected to N43, and the source connected to the source of the twenty seventh PMOS transistor. For the twenty seventh PMOS transistor, the drain is connected to the source of the twenty sixth PMOS transistor, the gate is connected to N43, and the source connected to the drain of the twenty eighth PMOS transistor. For the twenty eighth PMOS transistor, the drain is connected to the source of the twenty seventh PMOS transistor, the gate is connected to N43, and the source connected to the drain of the twenty nineth PMOS transistor. For the twenty nineth PMOS transistor, the drain is connected to the source of the twenty eighth PMOS transistor, the gate is connected to node N43, and the source connected to node N7.

### EXAMPLES

The following describes exemplary implementations, test results, etc. of embodiments disclosed herein.

The examples section presents a wireless application-specific integrated circuit (ASIC), operating with the MagSonic modality using one magnetoelectric (ME) transducer for neural stimulation and recording. The ASIC integrates a bridge circuit that forms both power management and data transmitter with voltage doubling, rectification, regulation, and over voltage protection, a biphasic AC stimulator with high voltage tolerance and direct external control simplifying downlink complexities and on-chip processing overhead, an active charge balancing circuit adjusting the duration of second stimulation phase, and a continuous neural recording and uplink communication. The prototype MagSonic ASIC was fabricated in a 180 nm standard CMOS process (2×1.75 mm² total area) and requires only one ME transducer and an external storage capacitor to operate. In measurements, a bar shaped millimeter-scale ME transducer (5.1×2.29×1.69 mm³) with length mode operation at 330 kHz was used to power the ASIC, achieving up to 8.1 mW of received power at 40 mm depth. The biphasic AC stimulator occupying only 0.027 mm² of active chip area provided 6.6 V (2×V_{DD}) tolerance (using 3.3 V transistors) with residual electrode voltage of <50 mV. The amplified signals were converted into time using an analog-to-time converter and transmitted at a data rate of 186.2 kbps (<10⁻³ BER) using the ME transducer's thickness mode frequency (1.66 MHz). Animal experiment results demonstrate the feasibility of ASIC's direct AC stimulation.

Miniature implantable medical devices (IMDs) with neural stimulation and recording capability, enabled by integrated circuit technology and advanced wireless power and data interrogation methods, pave the way for more localized and targeted treatment options. Fully wireless millimeter (mm) scale IMDs can minimize invasiveness and extend operational lifespan by eliminating the need for batteries. Exclusion of batteries also helps in reducing the number of surgeries needed for replacement and potential toxicity due to the battery leakage or heating inside the body. State-of-the-art technologies that exist for wireless power transfer to mm-scale IMDs include inductive, ultrasound (US) and magnetoelectric (ME) modalities. Efficient inductive links for mm-scale IMDs need to operate at high frequencies (100s of MHz), which suffer from limited external power transfer due to IEEE safety restrictions and high tissue absorption.

The US links achieve large received power, particularly at deep sites inside the tissue, while the ME links offer high tolerance to misalignment for wireless powering of mm-scale IMDs. But, ME-powered IMDs still have limited power particularly at deep sites and suffer from low uplink data rates as they use low frequency magnetic fields (MFs). For instance, has reported a 6 mm² ME film harvesting 3.8 mW at 30 mm depth (will be lesser at deeper sites), and has reported a 10 mm² ME achieving 2.7 mW at 50 mm depth in air (which can reduce when ME transducer is directly loaded with tissue). Some conventional system have achieved a limited uplink data rate of 17.73 kbps. While US links achieve higher power at deep sites and offer relatively higher data rates, they are greatly affected by misalignment.

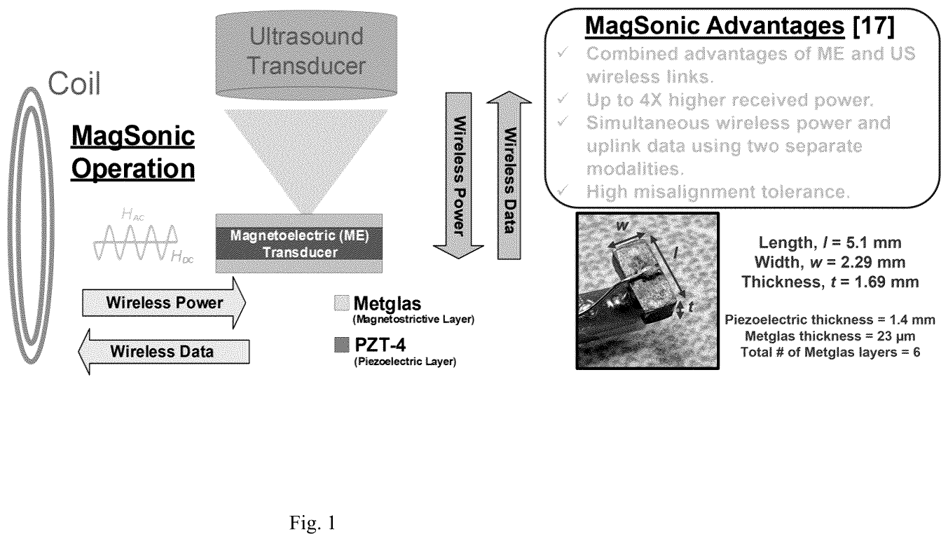

Recently, we have introduced a hybrid magnetic-ultrasonic (MagSonic) interrogation method that utilizes both ME and US modalities using a single ME transducer at the IMD site. As the ME transducer responds to both MF and US, the combined advantages outweigh the drawbacks of using a single (ME or US) modality. FIG. 1 shows a block diagram describing the MagSonic operation of the ME transducer, which is fabricated by sandwiching a piezoelectric layer in between one or several magnetostrictive layers. MagSonic link requires a wearable unit with a transmitter (Tx) coil and US transducer to interrogate an implant with an ASIC and ME transducer. In brief, the low-frequency MF generated by the external Tx coil is converted into an electric voltage by the direct ME effect, and the US energy generated by an external Tx US transducer is converted into an electric voltage via the direct piezoelectric effect of the piezoelectric layer in the ME transducer. For the uplink data communication, voltage is applied to the ME transducer, generating MFs and US waves based on the converse ME and converse piezoelectric effects, respectively, which are detected by an external receiver (Rx).

FIG. 1 also summarizes some key advantages of the MagSonic interrogation method, including up to 4-fold higher received power via MF and US modalities, simultaneous wireless power reception and uplink data communication using two distinct modalities with minimal interference, and higher tolerance to misalignments. Given these advantages, the present technology provides an application-specific integrated circuit (ASIC) developed specifically for the MagSonic interrogation link. Previous ASIC implementations with an ME transducer as a power receiver (and data transmitter) have achieved low received power (3.8 mW at 30 mm depth) or limited uplink data rates (17.73 kbps at 30 mm depth).

The MagSonic ASIC in this paper includes several novel circuits for fully wireless neural stimulation and recording. These include 1) a single bridge circuit that forms an integrated power management and data Tx (IPMDT) where power management performs ME voltage doubling with rectification, regulation, and over voltage protection (OVP), and the data Tx doubles the voltage applied across the ME transducer for uplink data transfer; 2) a biphasic AC stimulator with voltage tolerance up to 2×V_{DD}=6.6 V (V_{DD}: power supply of 3.3 V) directly controlled by the external unit simplifying downlink communication complexities (offering great flexibility in the selection of the stimulator's parameters with high resolution) and on-chip processing overhead; 3) an active charge balancing circuit that adjusts the duration of the second stimulation phase to ensure minimal residual charge on electrodes; 4) continuous neural recording and uplink communication with analog to time conversion using MFs or US waves. This work presents the first ASIC implementation of both MagSonic and direct AC stimulation, where the ME transducer's voltage is directly applied to the nerve, which has previously been shown to be effective.

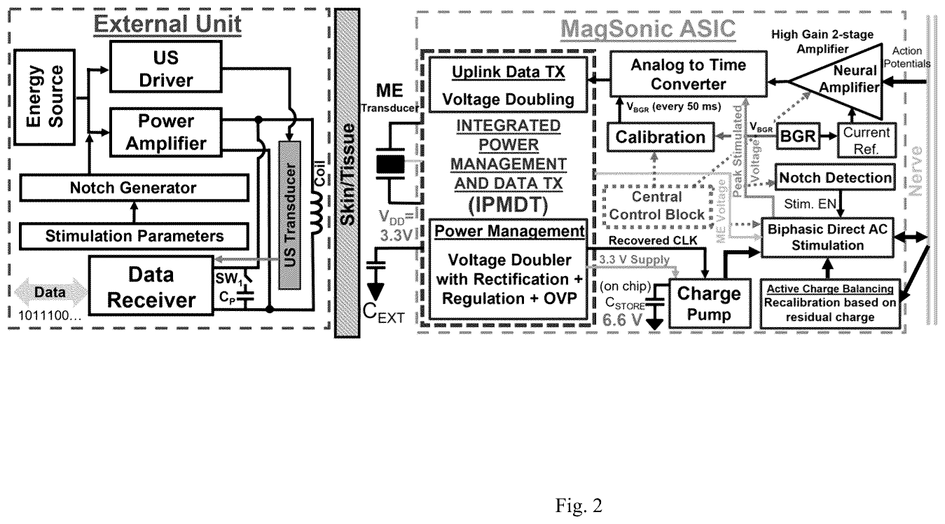

FIG. 2 shows the simplified block diagram of the wireless MagSonic system, including the architecture of the MagSonic ASIC. This ASIC is developed particularly for neural stimulation and recording applications. The ASIC has been designed in a 180 nm standard CMOS process, in which the maximum allowed voltage across any two terminals of the transistor is 3.3 V (V_{DD}) to prevent transistor overstress and breakdown. This process also supports deep-nwell NMOS transistors, which helps to separate the transistor's body and substrate terminals'potential.

The ASIC requires only two external components, a magnetoelectric transducer (ME) and a storage capacitor (C_{EXT}) for full MagSonic operation. The ASIC's key blocks include the IPMDT for power management (regulated voltage doubler) and data transmission, biphasic AC stimulator, active charge balancing, neural recording low-noise amplifier, analog-to-time converter for uplink communication using MF or US, and a calibration unit that sends the fixed bandgap reference (BGR) voltage (V_{BGR}=1.2 V) once every ˜50 ms, aiding the external unit to perform calibrations in the presence of process-voltage-temperature (PVT) variations. The following describe each of these blocks in detail.

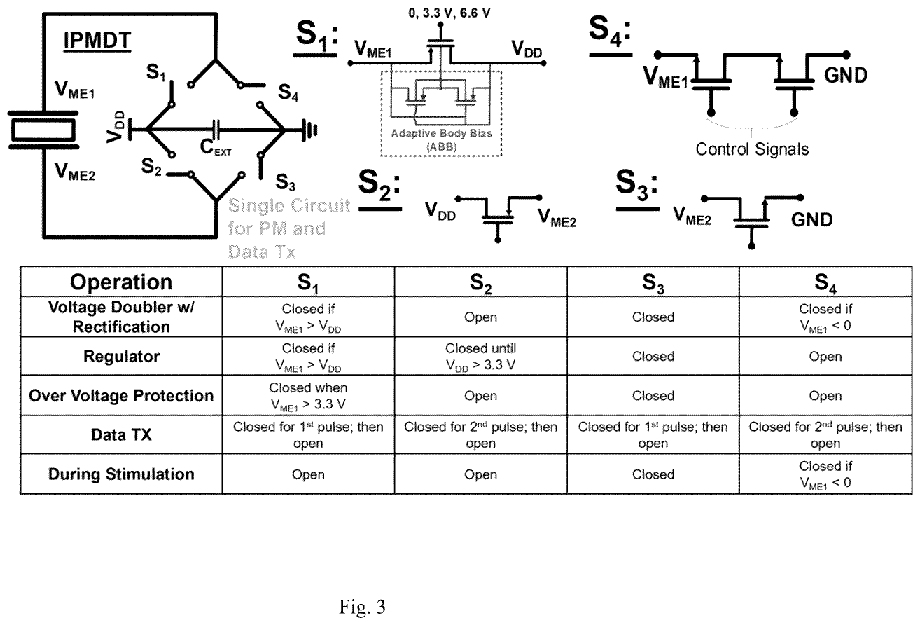

As MagSonic possesses the unique capability of simultaneous wireless power reception and uplink data communication, a unique circuit has been designed that seamlessly integrates the power management and data Tx into one. The circuit schematic of the IPMDT along with the switching operation table is shown in FIG. 3. The IPMDT has a bridge architecture responsible for both the chip's power management and uplink data transmission. In power-management configuration, voltage doubling, rectification, self-regulation, and OVP are all achieved by operating S₁₋₄ switches based on ME and C_{EXT} voltages. When the V_{ME1} voltage is greater than V_{DD}, and V_{DD} is less than the regulated voltage of 3.3 V, S₁ and S₃ are closed to transfer the charge from ME to C_{EXT}. During the negative power-carrier cycle, a zero-crossing detector (ZCD) is used to sense when V_{ME1} goes below 0 V to close S₃ and S₄, forming a voltage doubler. If C_{EXT} charges beyond 3.3 V, S₂ and S₃ are momentarily closed to discharge and achieve self-regulation (V_{DD}=3.3 V).

When the stimulation phase is not active and V_{ME1} goes beyond 3.3 V, ME is loaded by C_{EXT} for achieving OVP. Under different loading conditions, the optimal operation frequency of the ME transducer changes, which in turn helps with OVP. For uplink data transmission, S_{1,3} and S_{2,4} are closed and opened in alternate pairs, driving ME with V_{DD} and −V_{DD} (effectively doubling the voltage across ME to 2×V_{DD}, improving the communication reliability). The duration for which the switch pairs are closed is controlled by digital circuitry corresponding to ME's width or thickness mode resonance frequency.

As shown in FIG. 3, S₁ is a PMOS transistor whose gate voltage is either 0 V, 3.3 V, or 6.6 V depending on the operation mode and the ME voltage level (V_{ME1}). This is done to avoid the breakdown of 3.3 V transistors. When operating in IPMDT mode, its gate voltage is between 0 and 3.3 V. While the stimulator is active, it is between 3.3 V and 6.6 V to always keep this switch off. A level shifter is used to generate the 6.6 V control signal. A charge pump is used to generate the 6.6 V supply voltage on the chip. S₄ contains two series connected deep-nwell NMOS transistors (FIG. 3) to prevent breakdown when V_{ME1}>3.3 V during stimulation, in which S₄ is always off. The control signals for S₄ only change between 0 V and 3.3 V for all operation modes. S₁ utilizes the adaptive body biasing (ABB) technique[25], the schematic of which for S₁ is shown in FIG. 3. S₂ and S₃ are PMOS and NMOS switches, respectively.

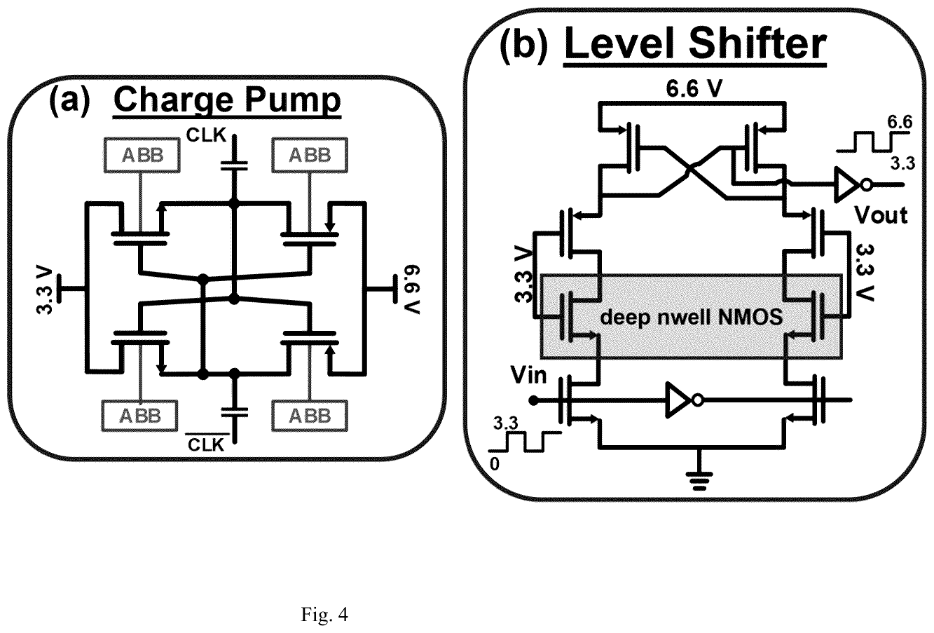

To enable stimulation voltage compliances of up to 6.6 V (2×V_{DD}), an on-chip charge pump (schematic shown in FIG. 4 with 100 pF pumping capacitors was designed to convert 3.3 V into 6.6 V. As the stimulation current is directly provided through the ME transducer, the charge pump output (6.6 V) is connected to an on-chip 1.2 nF storage capacitor, which is sufficient for driving the transistors (primarily switches) during high-voltage stimulation. The simulated charge pump's ripple at 1 MΩ load is 7.2 mV, with the highest simulated efficiency of ˜70% at 250 kΩ load. The charge pump clock at 165 kHz is recovered from the ME transducer's power carrier via digital circuitry.

A level shifter operating at 6.6 V is necessary to generate the 6.6 V control signal needed in the IPMDT and stimulator blocks. FIG. 4 shows the schematic of this level shifter, which is immune to voltage stress conditions ensuring reliable operation. As marked in FIG. 4, deep-nwell NMOS transistors are used in the cascode stage.

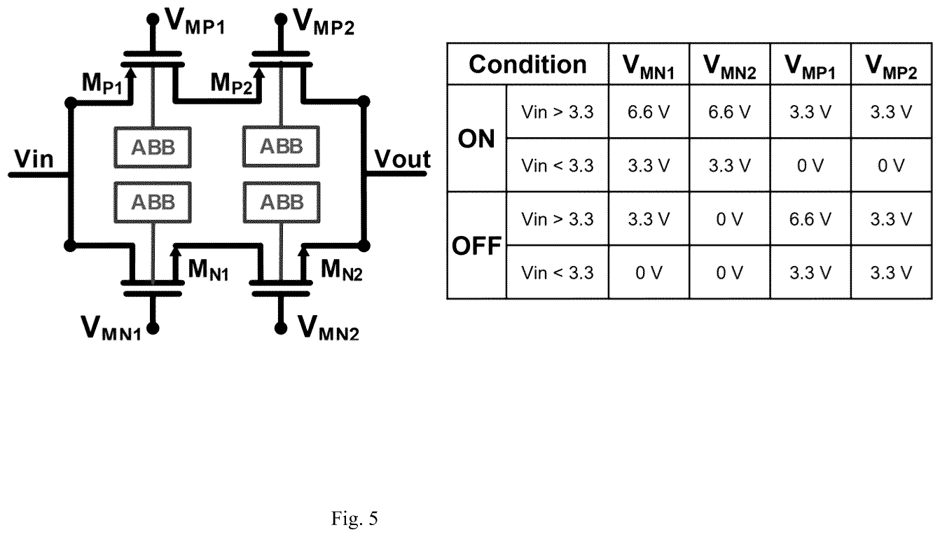

A high-voltage tolerant switch for the stimulator is achieved by two series connected NMOS and PMOS pairs as shown in FIG. 5. All four transistors utilized ABB. Based on the input voltage (Vᵢₙ) level, and the required operation of the switch ON/OFF), the 4 control voltages needed are summarized in the table of FIG. 5. Such an operation always ensures that the potential difference between any two terminals of transistors is always below 3.3 V. To minimize the loss across stimulation switches, the PMOS and NMOS transistors were sized as 630/0.3 and 260/0.35 (all in μm), respectively, to achieve a series ON resistance of 10 Ω (5 Ω individually).

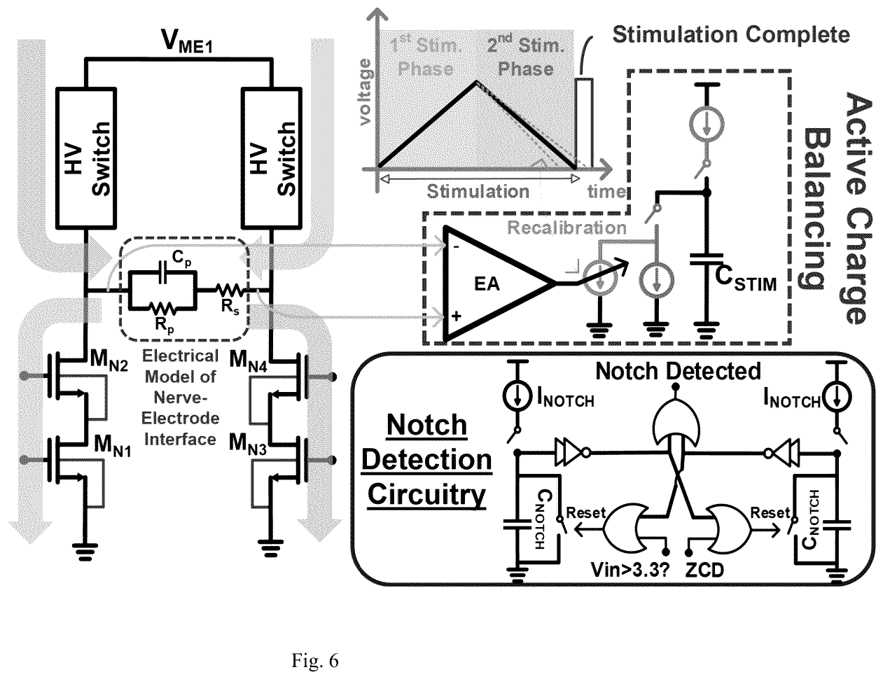

The MagSonic ASIC offers direct AC stimulation by connecting the ME transducer directly to the electrodes for the desired pulse duration[20]. While [20]conventional systems demonstrate the potential of AC-type stimulation, they suffers from several important limitations. They lack knowledge of stimulation strength, as Tx has no information about the actual voltage levels delivered during stimulation due to distance and alignment variations. They do not support charge balancing, which is essential for ensuring long-term safety and preventing tissue damage. Additionally, they do not have any recording capability for closed-loop operations. In contrast, the MagSonic chip disclosed herein addresses these limitations by enabling feedback-based monitoring and implementing active charge balancing for sinusoidal stimulation within a compact implantable platform, at the cost of slight increase of the implant size as a small chip is required. With MagSonic ASIC, two-phase stimulation, similar to bipolar stimulation, is achieved using an H-bridge architecture as shown in FIG. 6. The ASIC simplifies the downlink communication usually used for programming the stimulator parameters on the IMD. Instead, a notch in the power carrier informs the ASIC to begin the first stimulation phase. A second notch informs the ASIC to switch to the second stimulation phase.

This method offers considerable flexibility in configuring some stimulation parameters, such as amplitude, pulse width, and repetition rate, directly from the external unit. By shifting control complexity to the external side (where power and area constraints are significantly more relaxed), it also ensures power/area efficient stimulator circuity, high timing precision, and increased robustness, as it eliminates reliance on a relatively more complex downlink data stream that could be susceptible to channel interference. For instance, the notch detection circuitry occupies only 0.0005 mm² of active area and consumes 0.49 μW, enabled by the simplified data recovery architecture. Despite these advantages, the stimulation waveform is restricted to a sinusoidal shape, and it becomes challenging to independently stimulate multiple electrodes with different timing requirements.

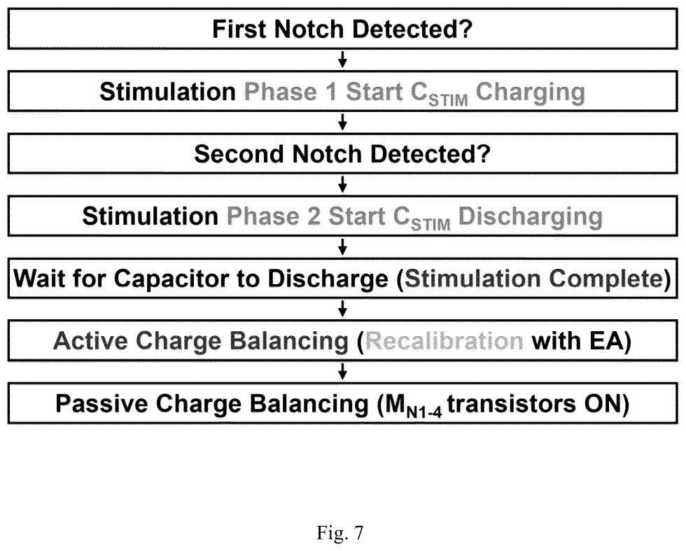

While active charge balancing by adjusting the duration of the second stimulation phase has been implemented in previous systems, the MagSonic ASIC extends this concept to direct AC stimulation, utilizing a sinusoidal signal derived directly from the power carrier. For this active charge balancing, at the beginning of the first stimulation phase, a 20 pF capacitor (C_{STIM}) starts to charge with a constant current of 20 nA. With this configuration, the maximum supported stimulation pulse duration is 3.3 ms, which is sufficient for most stimulation applications. During the second stimulation phase, C_{STIM} is discharged at a similar rate. When C_{STIM} is completely depleted, the stimulation is halted by the ASIC automatically, and the operation switches back to neural recording and data transmission. After the two-phase stimulation is completed, for a short period (360 μs), an error amplifier (EA) senses the remaining charge on the electrodes and recalibrates the discharge current needed for the next stimulation phase to minimize the residual voltage on the electrodes. The discharge phase includes a fixed 15 nA current along with a variable 10 nA current controlled by the error amplifier. During this recalibration phase, the peak stimulation voltage is sent back to the external unit via uplink communication for adjusting stimulation strength. At the end of this recalibration phase, transistors M_{N1-4} in the stimulator (FIG. 6) are activated for passive charge balancing. The stimulator operation with active and passive charge balancing is presented as a flowchart in FIG. 7.

To detect the notch in the power carrier, two C_{NOTCH} capacitors (each 1.8 pF) are constantly charged with I_{NOTCH}=150 nA, as shown in the FIG. 6 inset. Every time the ME voltage crosses 0 V or 3.3 V, these capacitors are reset. In the presence of a notch if either of these conditions are not met, the notch detector output is triggered. Approximately 20 power cycles are sufficient to trigger the notch detection.

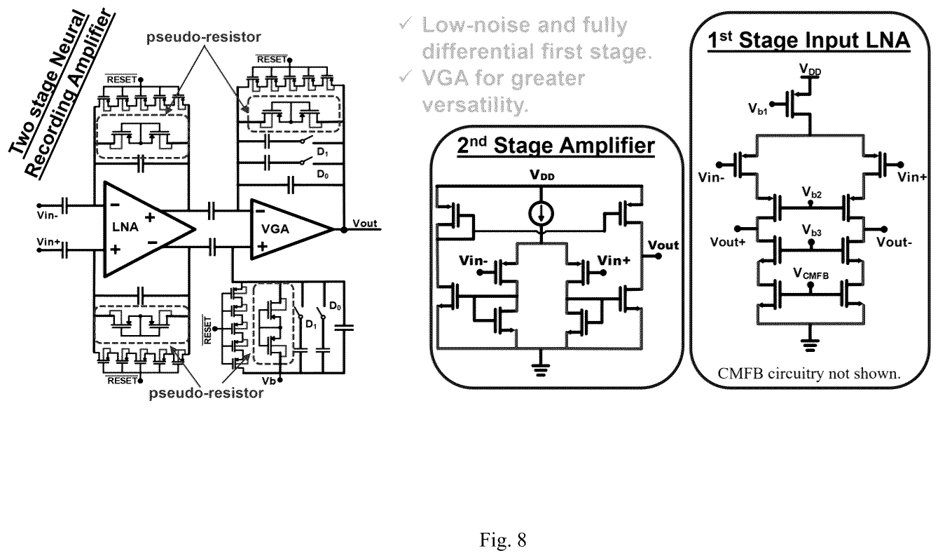

When the MagSonic ASIC is not in stimulation mode, it is always recording neural data and transmitting it wirelessly via uplink communication. FIG. 8 shows the schematic of the two-stage capacitively coupled low-noise amplifier for neural recording. The first stage telescopic op-amp with PMOS inputs and fully differential output ensures low noise and high common-mode rejection. Two diode-connected PMOS transistors form the pseudo-resistors, which are used to set the output DC level of these op-amps, eliminating the need for bulky resistors. The second stage of the neural amplifier uses a two-stage op-amp, as shown in FIG. 8. The feedback capacitors are used to set the gains of each stage. In the first stage, a capacitance ratio of 14.32 pF/0.13 pF (gain of 40.8 dB) is used, and in the second stage with variable gain, (4 different gains adjusted by selecting D₀ and D₁ values) the ratios 3.6 pF/(0.2 pF, 0.3 pF, 0.4 pF, or 0.5 pF) enabled 17.2-25.1 dB gain selection. D₀ and D₁ are one-time programmable bits that enable MagSonic ASIC to be more versatile. Operating at a 3.3 V supply, the total current consumption of this neural recording amplifier is simulated to be ˜4.5μA. The simulated input referred noise for this amplifier is ˜7 μVrms within ˜0.1 Hz-55 kHz (amplifier bandwidth: 0.4 Hz-3.2 kHz). The simulated differential input impedance of the amplifier is 11.17 MΩ at 1 kHz.

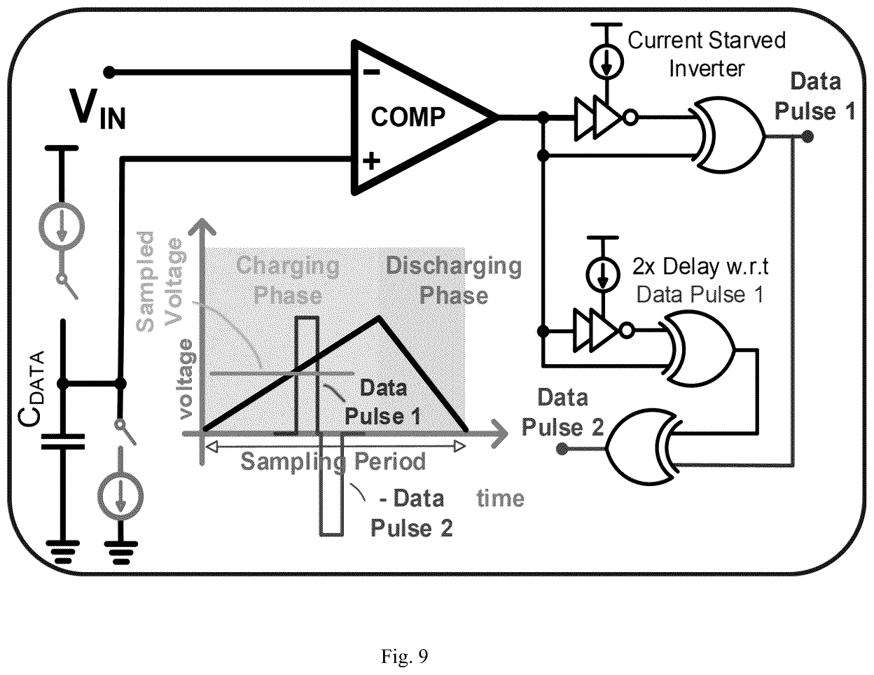

During the stimulation phase, to prevent saturation of the neural amplifier, it is reset by shorting the pseudo-resistors and maintaining the op-amps'outputs at their designated DC levels. The reset switches are several series-connected PMOS transistors, as shown in FIG. 8, to achieve large OFF resistance and better linearity. The neural amplifier output is converted into time information via an analog-time-converter (ATC) shown in FIG. 9. This time information is then relayed back to the external unit via uplink communication. In prior work we had demonstrated that pulse position modulation (PPM) significantly improves the uplink data rate of ME transducers operating at low frequencies. Therefore, the MagSonic ASIC incorporates the ATC, eliminating the need for a traditional analog-to-digital converter (ADC), which would incur higher power and area overhead while also limiting the achievable data rate.

In the ATC, the analog signal is compared with a constant ramping voltage across a capacitor (C_{DATA}) once every sampling cycle. When C_{DATA} voltage marginally exceeds the analog voltage, the comparator output flips and thus generates the data pulses to be transmitted. The data pulses are transmitted through the data Tx (within the IPMDT unit). Two data pulses are generated, as shown in FIG. 9, to effectively double the voltage across the ME transducer, further improving the uplink transmission range and reliability. The width of the data pulses is controlled by a current-starved inverter for operation in either width or thickness mode resonance frequency of the ME transducer being used for uplink communication.

By charging and discharging C_{DATA} of 1.1 pF with constant currents of 84 nA and 1.2 μA during fixed periods of ˜43.1μs and ˜3 μs, respectively, the designed sampling rate is ˜21.5 kSa/s. This is sufficient to reliably transmit most neural signals whose bandwidth is generally <10 kHz. To address PVT variations that can change the sampling rate, the ASIC includes an auto-calibration scheme. Upon power up, the ASIC transmits the fixed BGR voltage 8 times through the analog-to-time converter. Furthermore, the BGR voltage is sent once every 1024 samples. With this information, the external unit can re-calibrate itself on the fly to match the sampling rate of the ASIC.

To develop and demonstrate a complete MagSonic link with the ASIC, a mm-scale ME transducer was first fabricated and tested. Then, the ME transducer was integrated with the ASIC and an external C_{EXT} of 10 μF, and the whole link was tested.

Similar to our previous work in [19], the ME transducer (ME) was fabricated by sandwiching a 1.4 mm thick piezoelectric layer (PZT-4) between 6 magnetostrictive layers (Metglas; each layer being ˜23 μm thick), with 3 on each side. Adhesion was done using epoxy and conduction was ensured by applying a small amount of silver paste between the layers. The entire sample was cured under ˜3 kg weight at room temperature overnight. Two wires were then soldered on the top and bottom surfaces for electrical connections. As demonstrated in, ME was mounted on a PCB and coated with ˜10 μm Parylene-C biocompatible coating via the vapor deposition method. This coating provides electrical isolation as all measurements were performed in a water medium, as its acoustic impedance is similar to that of tissue (˜1.48 MRayl). An image of the fabricated bar-shaped 5.1×2.3×1.7 mm³ ME is shown in FIG. 1. It is worth noting that, while the operating frequency of the ME transducer is primarily determined by its length, its footprint (cross-sectional area) plays a critical role in enabling integration with planar electronics for mm-scale implants. In this work, the ME transducer thickness was experimentally optimized to receive the highest power using both MF and US modalities.

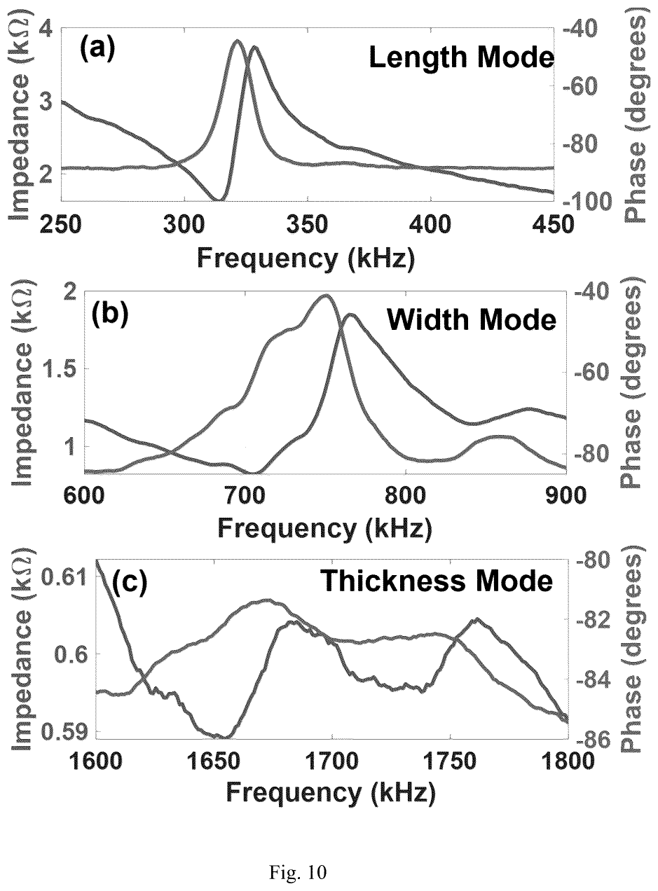

The fabricated ME was first characterized by measuring its impedance in water using a network analyzer. The frequency responses are shown in FIG. 10 for ME length, width, and thickness modes. The ME transducer is most efficient operating in the length mode, and thus, for power reception, it was operated at the length mode resonance of 330 kHz with measured impedance of 3.8 kΩ (graph a). The width and the thickness modes, occurring at a higher frequency, are more suitable for uplink data communication to achieve higher data rates. As shown in FIG. 10 (graphs b and c), the width and the thickness mode frequencies occurred at 761 kHz and 1.66 MHz, respectively. Multiple peaks occurring in the thickness mode frequency region may result from higher harmonics of the lower coupling modes. Therefore, the thickness mode resonance was identified as the frequency at which the Rx coil received the maximum voltage for a fixed voltage applied to the ME transducer. Compared to other modes, the phase response in this region shows a more capacitive behavior, likely due to parasitic capacitance between the parallel plates of the ME piezoelectric layer.

For wirelessly powering an IMD, it is crucial to adhere to safety limits. Under MagSonic operation, since ME transducer can be powered by MFs and US, both safety requirements need to be considered. The MF safety limit is governed by the IEEE safety guidelines, while US is governed by the Food and Drug Administration (FDA). We have provided a detailed safety discussion as well as the optimization of the Tx coil (for MF) and the Tx US transducer (for US), which were also adopted in this work. In brief, at operation frequency of fₚ=330 kHz, both electrostimulation or dosimetric reference limit (DRL) and SAR need to be considered. For DRL compliance, the maximum electric field must be less than 0.296×fₚ mV/m in tissue to prevent electrostimulation. For the optimized Tx planar coil [19] at fₚ=330 kHz, the highest electric field within the safety limit at the skin surface was 97.5 V/m, corresponding to H_{AC}=5.16 Oe limit at 40 mm depth inside the tissue. The specific absorption rate (SAR), pertaining to tissue heating, should also be below the 2 W/kg limit. The SAR value was simulated for each tissue layer, and the highest was 673 mW/kg for the muscle layer. Thus, low-frequency operation is limited by the DRL rather than the SAR. For the US transducer, the maximum US spatial-peak temporal-average intensity (I_{SPTA}) should be below 720 mW/cm² anywhere in the tissue.

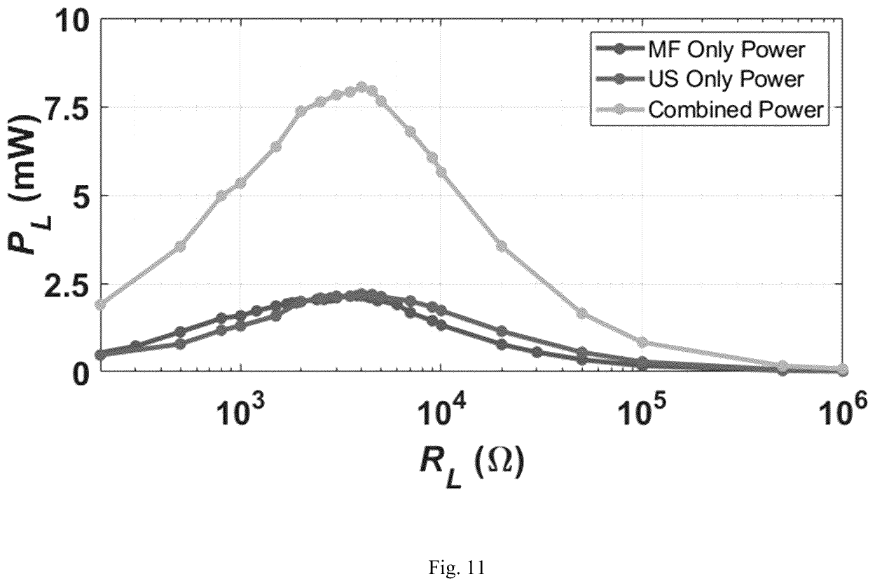

Based on these guidelines, ME's power reception capability was measured with MF, US, and MagSonic (MF+US) excitations. FIG. 11 shows the ME's P_{L} at various load resistances (R_{L}). At each R_{L}, a small range of frequencies was swept, and the highest corresponding P_{L} was recorded [14]. With 330 kHz, 5.16 Oe MF, ME achieved a P_{L} of 2.1 mW at optimal R_{L}=3.8 kΩ. Under 330 kHz, 625 mW/cm² US intensity, ME received P_{L}=2.2 mW at optimal R_{L}=4 kΩ. With MagSonic operation, the combined received P_{L} was 8.1 mW at R_{L}=4 kΩ. All these are reported at a Tx-Rx distance of 40 mm under safety limits.

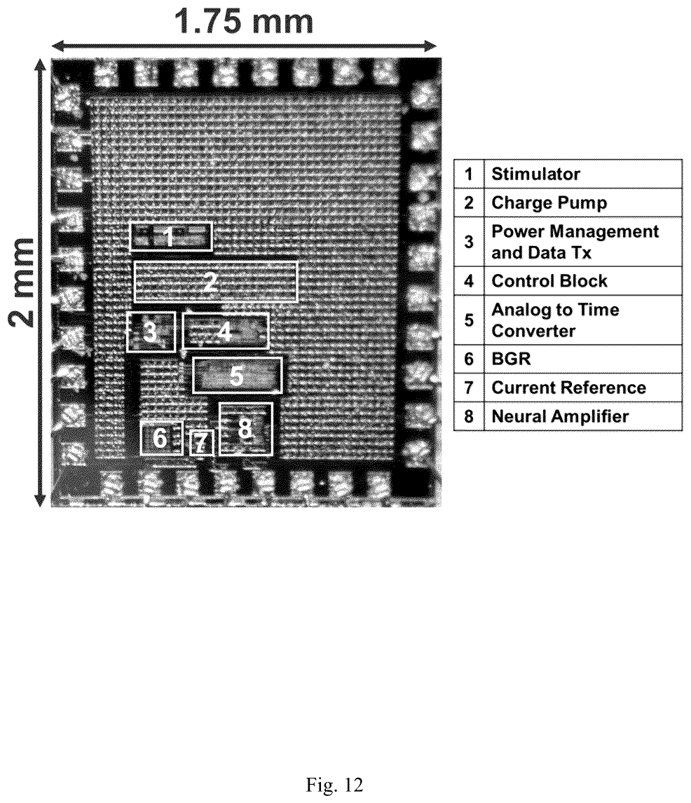

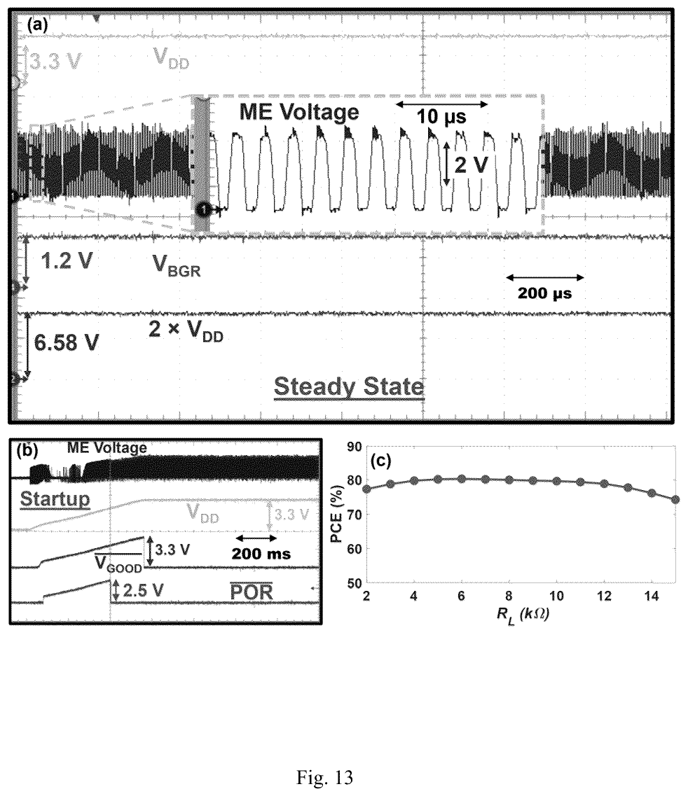

The MagSonic ASIC was fabricated in a 180 nm standard CMOS process. A die micrograph of the ASIC with a total area of 2×1.75 mm² is shown in FIG. 12. All measurements were conducted by mimicking a tissue environment with a water medium. FIG. 13 shows measured steady-state transient waveforms demonstrating the proper operation of the power management in ME voltage doubling and regulation at V_{DD}=3.3 V as well as generation of BGR voltage of 1.2 V and charge pump voltage of 6.58 V (˜2×V_{DD}). As shown in startup measurements in FIG. 13, it takes ˜550 ms for the ASIC to start up and reach regulated V_{DD} of 3.3 V. Upon power up, C_{EXT} starts to charge via the body diode leakage current in the power management (S₁ in FIG. 3). Once its voltage reaches 2.5 V, the inverted power-on-reset (POR) signal goes low, enabling the active voltage doubler with self-regulation. After this, power is stored in C_{EXT} at high efficiency.

The measured power conversion efficiency (PCE) of the voltage doubler (with only rectification) at different loading (R_{L}) conditions is shown in FIG. 13, with the highest achieved PCE of 80.2% at R_{L}=6 kΩ. In these measurements, the ME voltage was kept slightly below the voltage regulation limit of 3.3 V. For higher ME voltages, the PCE will drop similar to a linear regulator. The wireless power transfer efficiency of the magnetic link (from Tx coil input to ME output) was measured to be 0.01%. It is worth noting that the Tx coil generates peak MF of 5.16 Oe at 40 mm depth with 12.5 A peak current excitation, and the ME transducer generates 2.1 mW of power at this MF. At 40 mm depth, the US link power transfer efficiency was measured to be 0.08%.

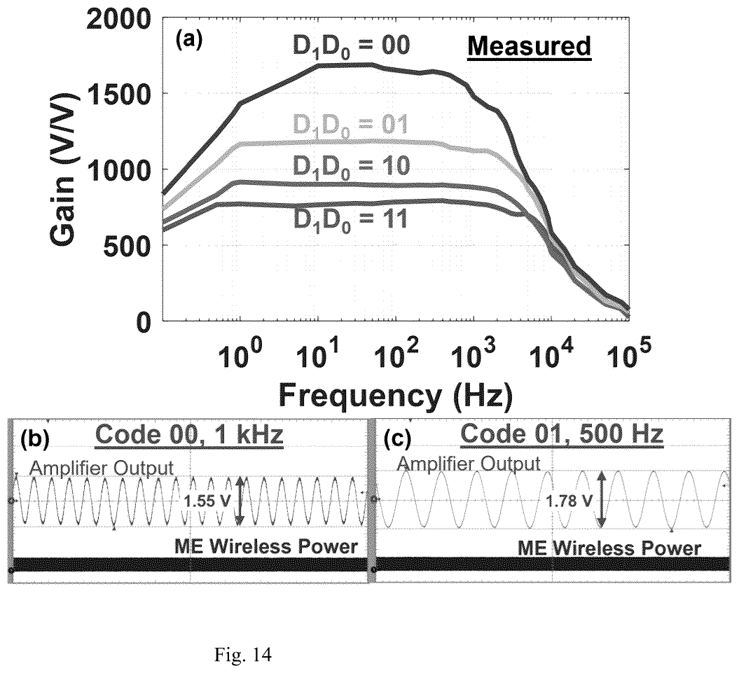

The measured frequency response of the neural amplifier for different gain settings (58-64.5 dB; externally controlled by two bits) is shown in FIG. 14. For the highest gain of 1685 V/V (64.5dB), the measured amplifier bandwidth was 0.4 Hz to 3.2 kHz. Measured transient waveforms with sinusoidal inputs of 950μV at 1 kHz and 1.5 mV at 500 Hz for different gain settings are also shown in FIG. 14 when the ASIC was wirelessly powered, indicating low noise and linear operation.

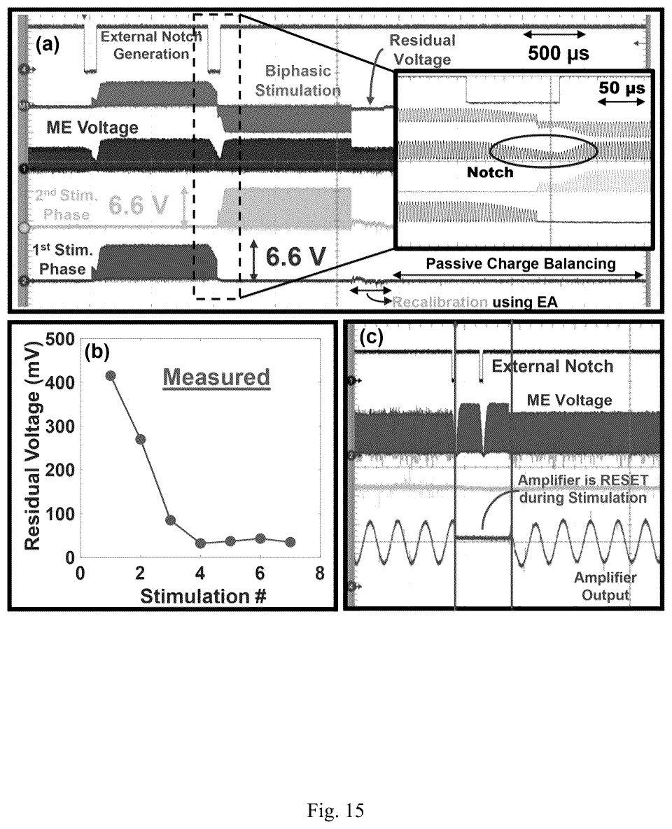

To verify the ASIC's stimulator operation, an electrical model of the nerve-electrode interface (FIG. 6: Cₚ=33 nF, Rₚ=5.08 MΩ, Rₛ=68 kΩ) was used in measurements. The measured transient waveforms in FIG. 15 show the notch generation, stimulation phase activation, switching from the 1^{st} stimulation phase to the 2^{nd} phase, and active/passive charge balancing operation (after 360 μs, the electrodes were shorted for passive discharge). The stimulation voltage reached up to 6.6 V in this measurement. As shown in FIG. 15, the active charge balancing circuit reduced the residual voltage to less than 50 mV within four stimulation cycles, which is considered safe. FIG. 15 also shows that the neural amplifier was reset during the stimulation phase (after first notch) when it was fed with a 1 mV, 1 kHz signal. Once the stimulation phase was complete, the amplifier immediately started amplifying the input signal.

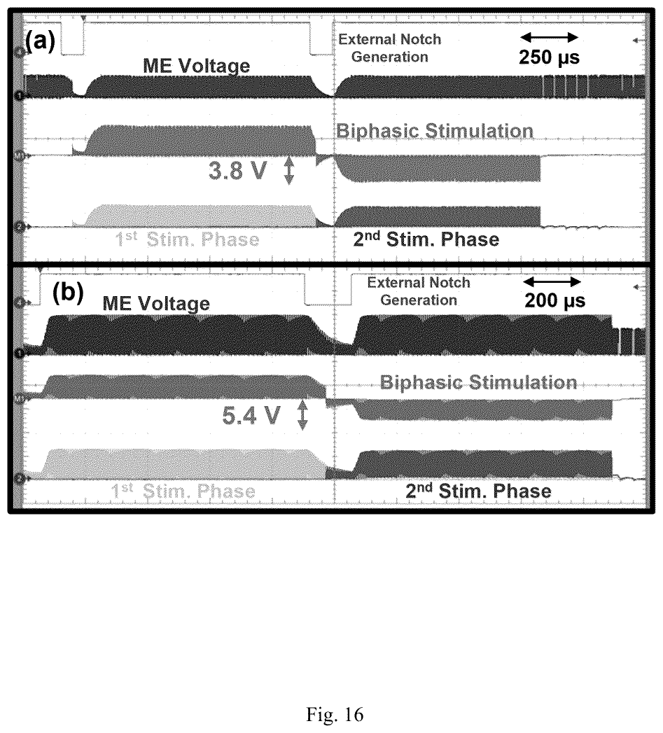

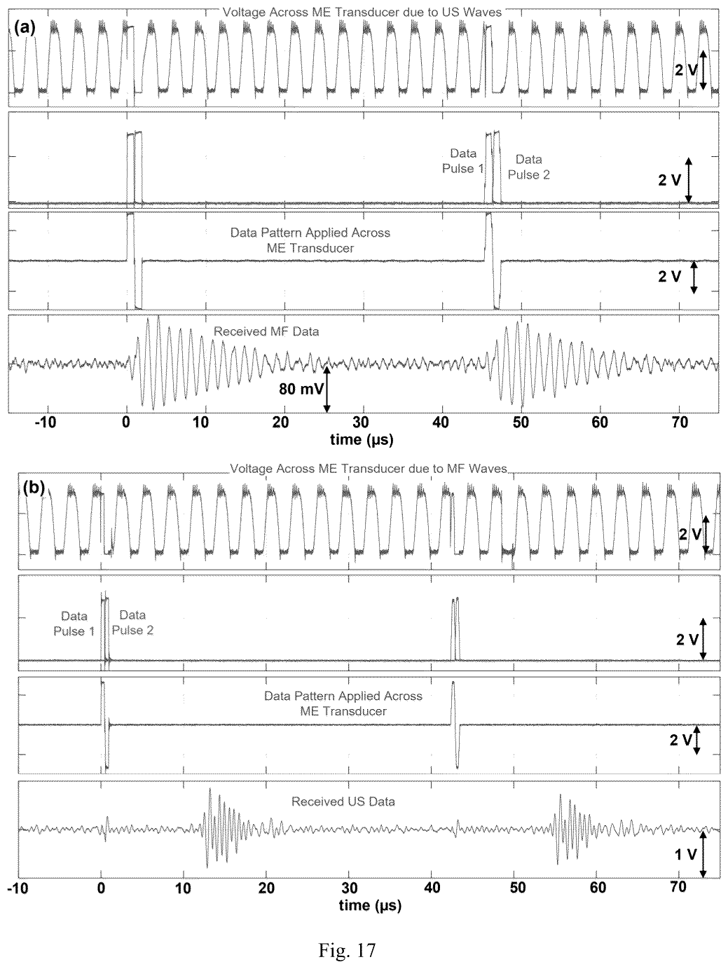

FIGS. 16 and 17 demonstrate the true operation of the MagSonic link with the ASIC. FIG. 16 shows measurement results demonstrating stimulation with single (330 kHz, MF=3 Oe-peak) and both (330 kHz, MF=3 Oe-peak and US=136 mW/cm²) modalities, effectively increasing the stimulation voltage from 3.8 V to 5.4 V. FIG. 17 shows measurement results demonstrating simultaneous power reception using US at ME's length mode resonance of 330 kHz and uplink data communication using MFs at ME's width mode resonance of 761 kHz and power reception (at 330 kHz) with MFs and simultaneous uplink communication with US at ME's thickness mode resonance of 1.66 MHz. All these measurements were performed at a Tx-Rx distance of 30 mm under safety guidelines. The detected data signals were amplified on the external end with a gain of 49 dB. It is worth noting that applying pulses to the ME transducer for data transmission can affect the received power in MagSonic, but our analysis shows a minimal effect as the data rate is low and data pulses are short.

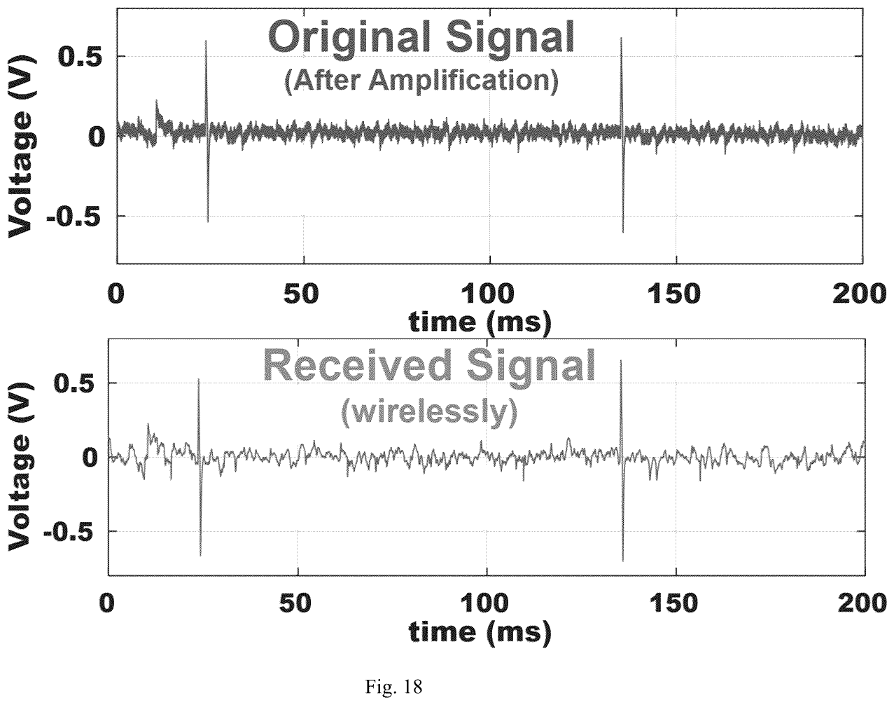

Typical neural action potentials or spikes recorded extra-cellularly have voltage amplitudes up to 500 μV in the 0.1 to 7 kHz band. Thus, for MagSonic wireless data link demonstration with the ASIC, the ASIC was wirelessly powered via MF, a synthetic neural signal with a peak voltage of ˜350 μV was fed to the neural amplifier input, and the data was wirelessly transmitted via US at 1.66 MHz. The US signals were detected on an oscilloscope whose sampling rate was 10 MSa/s (time resolution of 100 ns). The signal was reconstructed by finding the time difference between two consecutive received pulses. The original (after amplification) and reconstructed signals are shown in FIG. 18.

In this measurement, the ASIC produced a sample every 45.5 μs, thus yielding a data rate of 186.2 kbps (based on equations in with Δt=100 ns). As described in, the bit-error rate (BER) was measured to be <10⁻³ by verifying that at least 4.4 kilo samples were transmitted without an error. The BER could be lower, however, its estimation was limited by our measurement setup.

To demonstrate the effectiveness of the ASIC's direct AC stimulation, a preliminary animal experiment was conducted by stimulating the sciatic nerve of a mouse and measuring hindlimb movements. All procedures of animal studies were reviewed and approved by The Pennsylvania State University's IACUC.

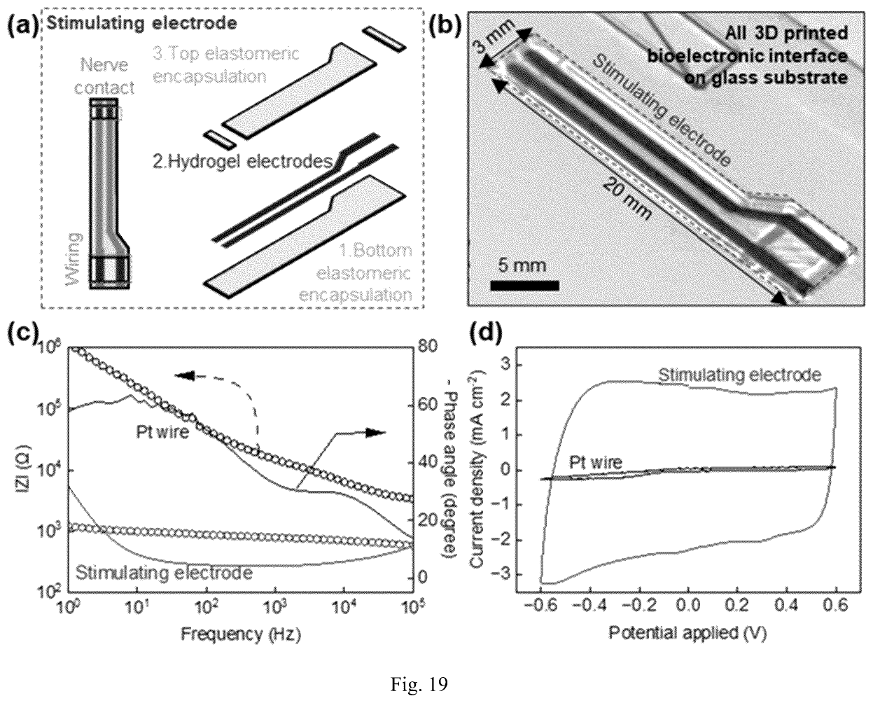

As shown in FIG. 19, the fabrication of the sciatic nerve electrode for stimulation (area: 3×20 mm²) was done with the direct ink writing (DIW)—based 3D printing following similar procedures with previous reports. First, the bottom encapsulation layer was printed using polydimethylsiloxane (PDMS) with a 100 μm nozzle. This layer was then cured at 125° C. for 15 minutes. Electrically conductive electrode layer was then printed using a poly (3,4-ethylenedioxythiophene): polystyrene sulfonate (PEDOT:PSS)/hydrophilic polyurethane (HPPU) hydrogel composite ink. After printing, the layer was air-dried for ˜5 minutes to ensure proper ink solidification. Finally, the top encapsulation layer, featuring electrode openings for direct contact with the sciatic nerve and external wire connections, was printed using the 100 μm nozzle and cured at 125° C. for 15 minutes.

The conductive polymer hydrogel comprising PEDOT:PSS blended with HPPU was used as the electrode material. This conductive hydrogel demonstrates a tissue-comparable Young's modulus of less than 1 MPa and exhibits high electrical conductivity exceeding 11 S/cm. In addition, it exhibits exceptional mechanical compliance with PDMS encapsulation (top and bottom layers). This conductive material is shown in stretchability up to 400% and a fracture toughness surpassing 3,300 J/m², rendering it highly suitable for in vivo implantation and neural interface applications. FIG. 19 shows an image of the fabricated stimulating electrode. The top PDMS encapsulation layer incorporates selectively exposed regions to create electrode openings for electrical contact with the nerve.

Electrochemical impedance spectroscopy was conducted to assess the interfacial impedance characteristics of the printed implantable electrode in a medium that mimics the ionic composition of in vivo animal models. For comparison, commercially available platinum (Pt) wire, commonly employed in implantable probes, was evaluated under identical experimental conditions. As shown in FIG. 19, the printed electrode exhibited impedance values below 1.3 kΩ across the frequency range of 1 Hz to 100 kHz. In contrast, Pt wire showed much higher impedance, rising sharply from ˜5 kΩ to over 1 MΩ as the frequency decreased from 100 kHz to 1 Hz.

Phase angle analysis in FIG. 19 revealed that the stimulation electrode consistently maintained phase angles below 20° across all frequencies, indicative of predominantly resistive behavior with minimal capacitive contribution. Conversely, the Pt wire exhibited phase angles of up to 60°, reflecting significant capacitive effects. The markedly lower impedance and reduced phase angles observed in the printed electrode suggest that the incorporation of conducting polymer hydrogels in the electrode layer effectively minimizes interfacial impedance and capacitive behavior, thereby enhancing signal fidelity for neural stimulation.

To further characterize their electrochemical performance, cyclic voltammetry (CV) measurements were performed on the stimulation electrode and Pt wire (FIG. 19). The printed electrode demonstrated a charge storage capacity of ˜26 mC/cm² that was over 40-fold higher than that of the Pt wire (˜0.6 mC/cm²). Together, these results underscore the superior electrochemical properties of the printed implantable electrode and its high performance for stimulation applications.

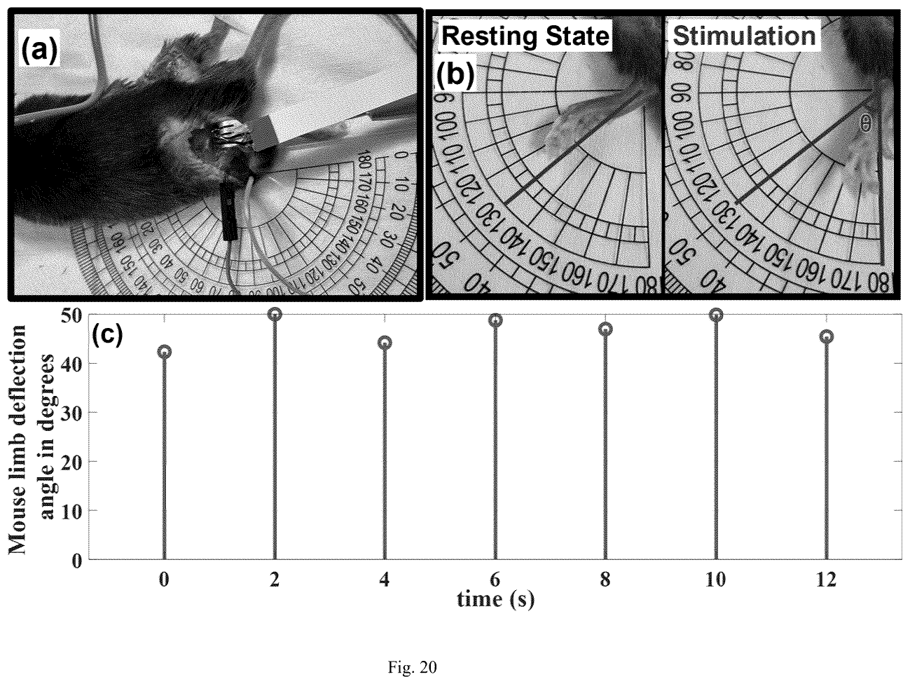

The fabricated electrode was surgically implanted onto the left hindlimb of the anesthetized adult male mouse (C57BL/6J, 9 weeks), weighing 28 grams. The mouse was anesthetized with 3% isoflurane delivered via inhalation, and anesthesia was maintained using a nose cone with 1-2% isoflurane in oxygen. As shown in FIG. 20, the electrode was carefully implanted around the sciatic nerve of the mouse. The 3D printed electrodes were electrically connected to the encapsulated metal wires using a conductive silver paste adhesive to ensure reliable electrical contact. One distal end of the metal wires was then connected to the ASIC board as the active stimulation site. For simplicity and to reduce the experiment variability, a reference wire electrode was implanted directly into the muscle tissue of the mouse and connected to the ASIC to provide the stimulation return path. The ASIC was powered wirelessly via MFs with direct AC stimulation applied to the sciatic nerve, and the hindlimb movement was recorded.

The stimulation pattern was a rectified 4.2 V AC voltage at 330 kHz with biphasic type stimulation (similar to FIG. 15 waveforms), pulse width of 1 ms, and 0.5 Hz (once every 2 s) repetition. FIG. 20 demonstrates the movement of the mouse hindlimb. For this particular stimulation specification, the mouse hindlimb moved by an average angle of ˜50° whenever the stimulation was active. FIG. 20 also shows the variation of this angle for a repeated stimulation pattern every 2 s.

This animal experiment demonstrates the effectiveness of the direct AC stimulation applied by connecting the rectified ME transducer AC voltage to the nerve in inducing electrical stimulation onto the nerve. It also demonstrates the MagSonic ASIC's ability to apply AC stimulation voltages up to 6.6 V (2×V_{DD}) compliance in a biphasic fashion controlled entirely by the external unit. To the best of our knowledge, this is the first demonstration of the effectiveness of direct AC neural stimulation with a fully wireless ASIC.

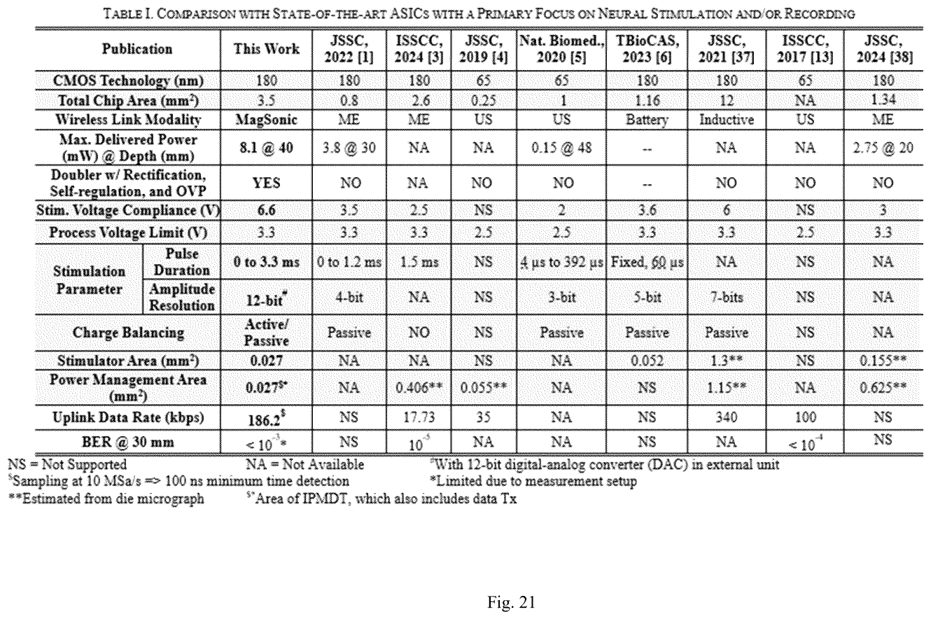

FIG. 21 benchmarks the MagSonic ASIC with state-of-the-art ASICs that utilize wireless interrogation for neural stimulation applications where the implant size is in the mm scale. The MagSonic ASIC particularly stands out in terms of maximum power delivery, stimulation compliance voltage range supporting 6.6 V which is two times the highest voltage supported by the process (V_{DD}); a new active charge balancing scheme that dynamically adjusts the stimulation time period to reduce the residual charge on the nerve; the smallest reported stimulator area and highest controllability of stimulation voltage and duration achieved by moving the complexity to the external unit; and a unique circuit that combines power management and data Tx for a fully wireless battery-less implant. As the chip uses MagSonic interrogation for wireless power and data communication, it inherently bolsters its high-received wireless power operating under safety limits, high data rate, and high misalignment tolerance benefits, as demonstrated in. This work has the highest reported uplink communication rate of 186.2 kbps utilizing a single ME transducer with a BER of <10⁻³, supporting simultaneous wireless power reception and uplink data transmission.

For the external unit, we envision a wearable, lightweight, battery-operated belt-like system. This unit would ideally incorporate arrays of coils and US transducers to enable multi-angle power and data transfer. If angled placement of coils and US transducers is not feasible (as illustrated in FIG. 1), co-planar placement is also effective for MagSonic operation, as demonstrated in our previous work. The MagSonic interrogation link is broadly applicable to various use cases. In particular, we aim to utilize it for closed-loop gastric slow-wave mapping and neural recording/stimulation, as both power budget and implantation depth are well within the mW and cm-range requirements. Additionally, the MagSonic platform is suitable for EMG recording and stimulation in limbs (arms or legs). The cylindrical geometry of the limbs is advantageous, allowing the belt-like Tx to wrap around the body part and provide multi-angle wireless links—fully leveraging the directional flexibility and robustness of the MagSonic technique.

While our ASIC implementation of the direct AC stimulator proved viable, offering low area, power, and complexity in the implant, its efficiency and safety are to be studied. In the future, the neural recording capabilities of the ASIC, along with direct stimulation with MagSonic operation, are to be studied in vivo.

A fully wireless ASIC was presented that utilizes the MagSonic interrogation technique for neural stimulation and recording applications. The ASIC leverages the full potential of magnetic field (MF) and ultrasound (US) modalities with a single magnetoelectric (ME) transducer at the implant site providing the best of both worlds. We presented an ASIC capable of 6.6 V (2×V_{DD}) stimulation voltage compliance, new active charge balancing technique, very high stimulation parameter controllability, smallest stimulator area of 0.027 mm², neural data recording with the on-chip amplifier and transmitter via uplink communication at the highest reported uplink data rate (186.2 kbps; 1.66 MHz carrier) using a single ME transducer, and a new unique circuit that combines the power management and data Tx. The ASIC was fabricated in a 180 nm standard CMOS process and requires one external ME transducer and one external capacitor for its operation. Through an animal experiment, we also demonstrated direct AC stimulation effectiveness by successfully inducing hindlimb movement of a mouse by sciatic nerve stimulation.

### REFERENCES

The following references are incorporated herein by reference in their entireties.
[1] Z. Yu et al., “Magnetoelectric bio-implants powered and programmed by a single transmitter for coordinated multisite stimulation,” IEEE J. Sol. St. Cir., vol. 57, no. 3, pp. 818-830, March 2022. https://doi.org/10.1109/JSSC .2021.3129993
[2] W. Kim et al., “Magnetoelectrics enables large power delivery to mm-sized wireless bioelectronics,” J. App. Phy., vol. 34, no. 9, September 2023. https://doi.org/10.1101/2023.09.01.555944
[3] Z. Yu et al., “33.6 A millimetric batteryless biosensing and stimulating implant with magnetoelectric power transfer and 0.9 pJ/b PWM backscatter,” 2024 IEEE Int. Sol. St. Cir. Conf. (ISSCC), 2024, pp. 551-553, February 2024. https://doi.org/10.1109/ISSCC49657.2024.10454271
[4] M. M. Ghanbari et al., “A sub-mm³ ultrasonic free-floating implant for multi-mote neural recording,” J. of Sol. St. Cir., vol. 54, pp. 3017-3030, September 2019. https://doi.org/10.1109/JSSC.2019.2936303
[5] D. K. Piech et al., “A wireless millimeter-scale implantable neural stimulator with ultrasonically powered bidirectional communication,” Nat. Biomed. Eng., vol. 4, pp. 207-222, February 2020. https://doi.org/10.1038/s41551-020-0518-9
[6] C. C. Hsieh, Y. H. Wu, and M. D. Ker, “Design of dual-configuration dualmode stimulator in low-voltage CMOS process for neuromodulation,” IEEE Trans. on Biomed. Cir. Sys., vol. 17, no. 2, pp. 273-285, April 2023. https://doi.org/10.1109/TBCAS.2023.3246164
[7] D. Zhou and E. Greenbaum, Implantable neural prostheses 1, New York, NY: Springer, 2009.
[8] K. Chen, Z. Yang, L. Hoang, J. Weiland, M. Humayun, and W. Liu, “An integrated 256-channel epiretinal prosthesis,” IEEE J. Solid State Cir., vol. 45, pp. 1946-1956, September 2010. https://doi.org/10.1109/JSSC.2010.2055371
[9] U. Jow and M. Ghovanloo, “Design and optimization of printed spiral coils for efficient transcutaneous inductive power transmission,” IEEE Trans. Biomed. Cir. Syst., vol. 1, no. 3, pp. 193-202, September 2007. https://doi.org/10.1109/TBCAS.2007.913130
[10] M. Kiani, U. Jow, and M. Ghovanloo, “Design and optimization of a 3-coil inductive link for efficient wireless power transmission,” IEEE Trans. Biomed. Cir. Syst., vol. 5, pp. 579-591, December 2011. https://doi.org/10.1109/TBCAS.2011.2158431
[11] A. Ibrahim and M. Kiani, “A figure-of-merit for design and optimization of inductive power transmission links for millimeter-sized biomedical implants,” IEEE Trans. Biomed. Cir. Syst., vol. 10, no. 6, pp. 1100-1111, December 2016. https://doi.org/10.1109/TBCAS.2016.2515541
[12] A. Ibrahim, M. Meng, and M. Kiani, “A comprehensive comparative study on inductive and ultrasonic wireless power transmission to biomedical implants,” IEEE Sens. J., vol. 18, pp. 3813-3826, May 2018. https://doi.org/10.1109/JSEN.2018.2812420
[13] T. C. Chang, M. L. Wang, J. Charthad, M. J. Weber, and A. Arbabian, “27.7 A 30.5 mm³ fully packaged implantable device with duplex ultrasonic data and power links achieving 95 kb/s with <10⁻⁴ BER at 8.5 cm depth,” IEEE Int. Sol. St. Cir. Conf. (ISSCC), 2017, pp. 460-461, February 2017. https://doi.org/10.1109/ISSCC.2017.7870460
[14] S. Hosur et al., “A comprehensive study on magnetoelectric transducers for wireless power transfer using low-frequency magnetic fields,” IEEE Trans. Biomed. Cir. Syst., vol. 15, pp. 1079-1092, October 2021. https://doi.org/10.1109/TBCAS.2021.3118981
[15] S.K. Karan, S. Hosur, Z. Kashani, H. Leng, A. Vijay, R. Sriramdas, K. Wang, B. Poudel, A. Patterson, M. Kiani, and S. Priya, “Magnetic field and ultrasound induced simultaneous wireless energy harvesting,” Energy and Env. Sci., vol. 17, pp. 2129-2144, January 2024. https://doi.org/10.1039/D3EE03889K
[16] IEEE standard for safety levels with respect to human exposure to electric, magnetic, and electromagnetic fields, 0 Hz to 300 GHz, IEEE Std C95.1-2019, pp. 1-312, October 2019. https://doi.org/10.1109/IEEESTD.2019.8859679
[17] S. Hosur, K. Nugmanuly, and M. Kiani, “A comparative study of advanced techniques for wireless power transfer to miniature medical implants,” IEEE Int. Sym. On Cir. & Syst. Conf. (ISCAS), London, U.K., May 2025. https://doi.org/10.1109/ISCAS56072.2025.11043881
[18] S. Hosur, S. K. Karan, S. Priya, and M. Kiani, “A comparative study on wireless power transfer to millimeter-scale biomedical implants with ultrasound and magnetoelectric technologies,” 2023 IEEE Biomed. Cir. and Sys. Conf. (BioCAS), Toronto, ON, Canada, pp. 1-5, October 2023. https://doi.org/10.1109/BioCAS58349.2023.10388751
[19] S. Hosur, Z. Kashani, S. K. Karan, S. Priya, and M. Kiani, “MagSonic: Hybrid magnetic-ultrasonic wireless interrogation of millimeter-scale biomedical implants with magnetoelectric transducer,” IEEE Trans. Biomed. Cir. Syst., vol. 18, no. 2, pp. 383-395, April 2024. https://doi.org/10.1109/TBCAS.2023.3334166
[20] J. C. Chen et al., “Self-rectifying magnetoelectric metamaterials for remote neural stimulation and motor function restoration,” Nat. Mater., no. 23, pp. 139-146, January 2024. https://doi.org/10.1038/s41563-023-01680-4
[21] M. Meng et al., “An Ultrasonically Powered Wireless System for In Vivo Gastric Slow-Wave Recording,” 41^{st} Ann. Int. Conf. of the IEEE Eng. in Med. and Bio. Soc. (EMBC), Berlin, Germany, pp. 7064-7067, July 2019. https://doi.org/10.1109/EMBC.2019.8857243
[22] S. Priya et al., “A Review on Piezoelectric Energy Harvesting: Materials, Methods, and Circuits,” Eng. Har. and Sys., vol. 4, no. 1, pp. 3-39, February 2017. https://doi.org/10.1515/ehs-2016-0028
[23] B. Serneels, M. Steyaert, and W. Dehaene, “A high speed, low voltage to high voltage level shifter in standard 1.2V 0.13 μm CMOS,” IEEE Int. Conf. on Elec., Cir. Sys., 2006, pp. 668-671, December 2006. https://doi.org/10.1109/ICECS.2006.379877
[24] M. D. Ker, S. L. Chen, and C. S. Tsai, “Design of charge pump circuit with consideration of gate-oxide reliability in low-voltage CMOS processes,” IEEE J. Sol. St. Cir., vol. 41, no. 5, pp. 1100-1107, May 2006. https://doi.org/10.1109/JSSC.2006.872704
[25] H. Lee and M. Ghovanloo, “A high frequency active voltage doubler in standard CMOS using offset-controlled comparators for inductive power transmission,” IEEE Trans. Biomed. Cir. Syst., vol. 7, no. 3, pp. 213-224, June 2013. https://doi.org/10.1109/TBCAS.2012.2198649
[26] F. Shahrokhi, K. Abdelhalim, D. Serletis, P. L. Carlen, and R. Genov, “The 128-channel fully differential digital integrated neural recording and stimulation interface,” IEEE Trans. Biomed. Cir. Sys., vol. 4, no. 3, pp. 149-161, June 2010. https://doi.org/10.1109/TBCAS.2010.2041350
[27] R. R. Harrison and C. Charles, “A low-power low-noise CMOS amplifier for neural recording applications,” IEEE J. Sol. St. Cir., vol. 38, no. 6, pp. 958-965, June 2003. https://doi.org/10.1109/JSSC.2003.811979
[28] S. Hosur, S. K. Karan, S. Priya and M. Kiani, “Packaging methods for magnetoelectric transducers used as wireless power receivers,” IEEE Biomed. Cir. Sys. Conf. (BioCAS), pp. 6-10, October 2022. https://doi.org/10.1109/BioCAS54905.2022.9948603
[29] H. Azhari, Basics of biomedical ultrasound for engineers Hoboken, NJ, 2010.
[30] Marketing clearance of diagnostic ultrasound systems and transducers, FDA, 2019.
[31] F. Kölbl, A. Zbrzeski, E. Syed, S. Renaud, and N. Lewis, “In vivo electrical characterization of deep brain electrode and impact on bio-amplifier design,” IEEE Biomed. Cir. Sys. Conf. (BioCAS), pp. 210-213, November 2010. https://doi.org/10.1109/BIOCAS.2010.5709608
[32] U. Shin et al., “NeuralTree: A 256-channel 0.227-μJ/class versatile neural activity classification and closed-loop neuromodulation SoC,” IEEE J. Sol. St. Cir., vol. 57, no. 11, pp. 3243-3257, November 2022. https://doi.org/10.1109/JSSC.2022.3204508
[33] S. Hosur, S. K. Karan, S. Priya, and M. Kiani, “Short-range communication for small biomedical implants using magnetoelectric effect,” IEEE Int. Sym. on Cir. Syst. Conf. (ISCAS), pp 1-5, May 2023. https://doi.org/10.1109/ISCAS46773.2023
[34] M. Momin et al., “3D-printed flexible neural probes for recordings at single-neuron level,” Device, vol. 2, no. 12, December 2024. https://doi.org/10.1016/j. device.2024.100519
[35] S. Ahmed et al., “Stick-and-play bioadhesive hairlike electrodes for chronic EEG recording on human,” npj Biomed. Innov., vol. 2, no. 9, March 2025. https://doi.org/10.1038/s44385-025-00009-x
[36] T. Zhou et al., “3D printable high-performance conducting polymer hydrogel for all-hydrogel bioelectronic interfaces,” Nat. Mater., no. 22, pp. 895-902, July 2023. https://doi.org/10.1038/s41563-023-01569-2
[37] S. H. Wang et al., “Design of a bone-guided cochlear implant microsystem with monopolar biphasic multiple stimulations and evoked compound action potential acquisition and its in vivo verification,” IEEE J. Sol. St. Cir., vol. 56, no. 10, pp. 3062-3076, October 2021. https://doi.org/10.1109/JSSC.2021.3087629
[38] W. Wang et al., “Omnidirectional wireless power transfer for millimetric magnetoelectric biomedical implants,” IEEE J. Sol. St. Cir., vol. 59, no. 11, pp. 3599-3611, November 2024. https://doi.org/10.1109/JSSC.2024.3464533
F. T. Alrashdan et al., “Wearable wireless power systems for ‘ME-BIT’ magnetoelectric powered bio implants,” J. Neural Eng., vol. 18, July 2021. https://doi.org/10.1088/1741-2552/ac1178

It should be understood that the disclosure of a range of values is a disclosure of every numerical value within that range, including the end points. It should also be appreciated that some components, features, and/or configurations may be described in connection with only one particular embodiment, but these same components, features, and/or configurations can be applied or used with many other embodiments and should be considered applicable to the other embodiments, unless stated otherwise or unless such a component, feature, and/or configuration is technically impossible to use with the other embodiment. Thus, the components, features, and/or configurations of the various embodiments can be combined together in any manner and such combinations are expressly contemplated and disclosed by this statement.

It will be apparent to those skilled in the art that numerous modifications and variations of the described examples and embodiments are possible considering the above teachings of the disclosure. The disclosed examples and embodiments are presented for purposes of illustration only. Other alternate embodiments may include some or all of the features disclosed herein. Therefore, it is the intent to cover all such modifications and alternate embodiments as may come within the true scope of this invention, which is to be given the full breadth thereof.

It should be understood that modifications to the embodiments disclosed herein can be made to meet a particular set of design criteria. Therefore, while certain exemplary embodiments of the compositions, materials, apparatuses, systems, and methods of using and making the same disclosed herein have been discussed and illustrated, it is to be distinctly understood that the invention is not limited thereto but may be otherwise variously embodied and practiced within the scope of the following claims.

What is claimed is:

1. An implant stimulation and recording system, comprising:
a receiver;
an interrogator comprising a transmitter coil and an ultrasound transducer;
an implant comprising an integrated circuit and a magnetoelectric transducer;
wherein low-frequency magnetic fields are generated by the transmitter coil and converted into an electric voltage by the magnetoelectric transducer;
wherein ultrasound energy is generated by the transducer and converted into an electric voltage by the magnetoelectric transducer; and
wherein, when voltage is applied to the magnetoelectric transducer, the magnetoelectric transducer generates magnetic fields and ultrasound waves, wherein the receiver detects the magnetic fields and ultrasound waves generated by the magnetoelectric transducer.

2. The implant stimulation and recording system of claim 1, wherein:
the implant includes a storage capacitor.

3. The implant stimulation and recording system of claim 1, wherein:
the integrated circuit includes: an integrated power management and data transmitter (IPMDT); a biphasic alternating current stimulator, an active charge balancer circuit, a recording low-noise amplifier, an analog-to-time converter configured for uplink communication using magnetic fields and ultrasound waves, and a calibration unit.

4. The implant stimulation and recording system of claim 3, wherein:
the IPMDT is connected to a first voltage output (VME1) of the magnetoelectric transducer and a voltage output (VME2) of the magnetoelectric transducer; and
the IPMDT includes a bridge circuit comprising a first branch (B1) with a first switch (S1), a second branch (B2) with a second switch (S2), a third branch (B3) with a third switch (S3), a fourth branch (B4) with a fourth switch (S4), and a storage capacitor (Cext).

5. The implant stimulation and recording system of claim 4, wherein:
B1 is connected to node N1 and node N2, B2 is connected to node N1 and node N4, B3 is connected to node N3 and node N4, and B4 is connected to node N2 and node N3;
N1 is connected to a positive power supply (VDD) and to Cext;
N3 is connected to ground (GND) and to (Cext);
S1 is a P-channel Metal-Oxide-Semiconductor (PMOS) transistor having a source connected to VME1, a gate, and a drain connected to VDD;
S2 is a PMOS transistor having a source connected to VME2, a gate, and a drain connected to VDD;
S3 is a N-channel Metal-Oxide-Semiconductor (NMOS) transistor having a source connected to VME2, a gate, and a drain connected to GND;
S4 includes a first NMOS transistor and a second NMOS transistor, wherein:
the first NMOS transistor has a source connected to VME1, a gate, and a drain connected to a source of the second NMOS transistor;
the second NMOS transistor has a source connected to the drain of the first NMOS transistor, a gate, and a drain connected to GND.

6. The implant stimulation and recording system of claim 3, wherein:
the biphasic alternating current stimulator includes a charge pump circuit, a level shifter circuit, and a high-voltage tolerant switch circuit.

7. The implant stimulation and recording system of claim 6, wherein:
the charge pump circuit comprises:
a first N-channel Metal-Oxide-Semiconductor (NMOS) transistor, a second P-channel Metal-Oxide-Semiconductor (PMOS) transistor, a third PMOS transistor, and a fourth NMOS transistor;
the first NMOS transistor includes a source connected to node N3, a gate connected to node N2, a drain connected to node N1;
the second PMOS transistor includes a source connected to node N4, a gate connected to node N2, a drain connected to node N3;
N3 is connected to a pumping capacitor Cp1 whose other end is connected to a first clock (CLK1);
the third PMOS transistor includes a source connected to node N4, a gate connected to node N3, a drain connected to node N2;
a second pumping capacity Cp2 is connected between a second clock (CLK2) and to N2; and
the fourth PMOS transistor includes a source connected to N2, a gate connected to N3, and a drain connected to N1.

8. The implant stimulation and recording system of claim 6, wherein:
the level shifter circuit, comprises:
a first P-channel Metal-Oxide-Semiconductor (PMOS) transistor, a second PMOS transistor, a third N-channel Metal-Oxide Semiconductor (NMOS) transistor, and a fourth NMOS transistor, a fifth NMOS transistor, a sixth NMOS transistor, a seventh PMOS transistor, and an eight PMOS transistor;
the first PMOS transistor includes a source connected to node N1, a gate connected to node N2, and a drain connected to node N3;
the second PMOS transistor includes a source connected to N3, a gate connected to node N4, and a drain connected to node N5;
the third NMOS transistor includes a drain connected to N5, a gate connected to N4, and a source connected to N6;
the fourth NMOS transistor includes a source drain to N6, a gate connected to node N7, and a source connected to node N8;
N7 is connected to a control signal (Vin), and N8 is connected to ground (GND);
the fifth NMOS transistor includes a drain connected to node N9, a gate connected to node N10, and a source connected to node N8;
N7 is connected to a first inverter (INV1);
the sixth NMOS transistor includes a drain connected to node N11, a gate connected to node N12, and a source connected to N9;
the seventh PMOS transistor includes a source connected to node N13, a gate connected to node N12, and a drain connected to N11; and
the eighth PMOS transistor includes a source connected to N1, a gate connected N2 and to a second inverter (INV2), and a drain connected to N13.

9. The implant stimulation and recording system of claim 6, wherein:
the high-voltage tolerant switch circuit comprises:
a first P-channel Metal-Oxide-Semiconductor (PMOS) transistor, a second PMOS transistor, a third N-channel Metal-Oxide-Semiconductor (NMOS) transistor, and a fourth NMOS transistor;
the first PMOS transistor includes a source connected to node N1, a gate connected to node N2, and a drain connected to node N3;
N2 is connected to a voltage control signal (VMP1);
the second PMOS transistor includes a source connected to N3, a gate connected to node N4, and a drain connected to node N5;
N4 is connected to voltage control signal (VMP2);
the third NMOS transistor includes a source connected to N7, a gate connected to node N6, and a drain connected to node N1;
N6 is connected to voltage control signal (VMN1);
the fourth NMOS transistor includes a source connected to N5, a gate connected to node N8, and a drain connected to N7; and
N8 is connected to voltage control signal (VMN2).

10. The implant stimulation and recording system of claim 3, wherein:
the biphasic alternating current stimulator circuit comprises:
a first high voltage switch connected to node N1 and node N2;
a second high voltage switch connected to N1 and node N3;
N1 is connected to a voltage source (VME1);
a first N-channel Metal-Oxide-Semiconductor (NMOS) transistor, a second NMOS transistor, a third NMOS transistor, and a fourth NMOS transistor;
the first NMOS transistor includes a drain connected to N2, a gate, and a source connected to node N4;
the second NMOS transistor includes a drain connected to N4, a gate, and a source connected to node N5;
N5 is connected to a first ground (GND1);
the third NMOS transistor includes a drain connected to N3, a gate, and a source connected to node N6;
the fourth NMOS transistor includes a drain connected to N6, a gate, and a source connected to node N7;
N7 is connected to a second ground (GND2).

11. The implant stimulation and recording system of claim 3, wherein:
the recording low-noise amplifier comprises:
a first op-amp (OP-AMP1), a second op-amp (OP-AMP2), a first circuit block, a second circuit block, a third circuit block, and a fourth circuit block;
OPAMP1 includes a first input connected to node N2, a second input connected to node N3, a first output connected to node N4, and a second output connected to node N5;
N2 is connected to a first capacitor (C1), and C1 is connected to node N1, wherein N1 is connected to a input voltage signal (Vin−);
N3 is connected to a second capacitor (C2), and C2 is connected to node N8, wherein N8 is connected to a input voltage signal (Vin+);
N4 is connected to a third capacitor (C3), and C3 is connected to node N6;
N5 is connected to a fourth capacitor (C4), and C4 is connected to node N7;
OP-AMP2 includes a first input connected to N6, a second input connected to N7, and an output connected to node N9;
the first circuit block comprises:
a fifth capacitor (C5) connected to node N2 and node N4;
a first P-channel Metal-Oxide-Semiconductor (PMOS) transistor, a second PMOS transistor, a third PMOS transistor, a fourth PMOS transistor, a fifth PMOS transistor, a sixth PMOS transistor, and a seventh PMOS transistor;
the first PMOS transistor includes a drain connected to node N2, a gate connected to node N13, and a source connected to N13;
the second PMOS transistor includes a drain connected to node N4, a gate connected to N13, and a source connected to N13;
each of the third, fourth, fifth, sixth, and seventh PMOS transistors includes a source, a gate, and a drain;
for the third PMOS transistor, the drain is connected to N12, the gate is connected to node N15, and the source connected to the drain of the fourth PMOS transistor;
for the fourth PMOS transistor, the drain is connected to the source of the third PMOS transistor, the gate is connected to node N15, and the source connected to the drain of the fifth PMOS transistor;
for the fifth PMOS transistor, the drain is connected to the source of the fourth PMOS transistor, the gate is connected to node N15, and the source connected to the drain of the sixth PMOS transistor;
for the sixth PMOS transistor, the drain is connected to the source of the fifth PMOS transistor, the gate is connected to node N15, and the source connected to the drain of the seventh PMOS transistor;
for the seventh PMOS transistor, the drain is connected to the source of the sixth PMOS transistor, the gate is connected to node N15, and the source connected to N4;
the second circuit block comprises:
a sixth capacitor (C6) connected to node N6 and node N17, wherein N17 is connected to N9;
a seventh capacitor (C7) connected to node N6 and node N17 via a first switch (D1), wherein N19 is connected to N17;
an eighth capacitor (C8) connected to node N6 and node N17 via a second switch (D2);
a ninth PMOS transistor, a tenth PMOS transistor, an eleventh PMOS transistor, a twelfth PMOS transistor, a thirteenth PMOS transistor, a fourteenth PMOS transistor, and a fifteenth PMOS transistor;
the ninth PMOS transistor includes a drain connected to node N6, a gate connected to node N23, and a source connected to N23, wherein N6 is connected to N17;
the tenth PMOS transistor includes a drain connected to node N24, a gate connected to N23, and a source connected to N23, wherein N17 is connected to N6;
each of the eleventh, twelfth, thirteenth, fourteenth, and fifteenth PMOS transistors includes a source, a gate, and a drain;
for the eleventh PMOS transistor, the drain is connected to N22, the gate is connected to node N25, and the source connected to the drain of the twelfth PMOS transistor;
for the twelfth PMOS transistor, the drain is connected to the source of the eleventh PMOS transistor, the gate is connected to node N25, and the source connected to the drain of the thirteenth PMOS transistor;
for the thirteenth PMOS transistor, the drain is connected to the source of the twelfth PMOS transistor, the gate is connected to node N25, and the source connected to the drain of the fourteenth PMOS transistor;
for the fourteenth PMOS transistor, the drian is connected to the source of the thirteenth PMOS transistor, the gate is connected to node N25, and the source connected to the drain of the fifteenth PMOS transistor;
for the fifteenth PMOS transistor, the drain is connected to the source of the fourteenth PMOS transistor, the gate is connected to node N25, and the source connected to N17;
the third circuit block comprises:
a ninth capacitor (C9) connected to node N3 and node N5;
a sixteenth PMOS transistor, a seventeenth PMOS transistor, an eighteenth PMOS transistor, a nineteenth PMOS transistor, a twentieth PMOS transistor, a twenty first PMOS transistor, and a twenty second PMOS transistor;
the sixteenth PMOS transistor includes a drain connected to node N3, a gate connected to node N29, and a source connected to N29;
the seventeenth PMOS transistor includes a drain connected to node N5, a gate connected to N29, and a source connected to N29;
each of the eighteenth, nineteenth, twentieth, twenty first, and twenty second PMOS transistors includes a source, a gate, and a drain;
for the eighteenth PMOS transistor, the drain is connected to N28, the gate is connected to node N31, and the source connected to the drain of the nineteenth PMOS transistor;
for the nineteenth PMOS transistor, the drain is connected to the source of the eighteenth PMOS transistor, the gate is connected to node N31, and the source connected to the drain of the twentieth PMOS transistor;
for the twentieth PMOS transistor, the drain is connected to the source of the nineteenth PMOS transistor, the gate is connected to node N31, and the source connected to the drain of the twenty first PMOS transistor;
for the twenty first PMOS transistor, the drain is connected to the source of the twentieth PMOS transistor, the gate is connected to node N31, and the source connected to the drain of the twenty second PMOS transistor;
for the twenty second PMOS transistor, the drain is connected to the source of the twenty first PMOS transistor, the gate is connected to node N31, and the source connected to N5;
the fourth circuit block comprises:
a tenth capacitor (C10) connected to node N7 and node N33;
an eleventh capacitor (C11) connected to node N32 and node N33 via a third switch (D3;
a twelfth capacitor (C12) connected to node N7 and node N33 via a fourth switch (D4);
a twenty third PMOS transistor, a twenty fourth PMOS transistor, a twenty fifth PMOS transistor, a twenty sixth PMOS transistor, a twenty seventh PMOS transistor, a twenty eighth PMOS transistor, and a twenty ninth PMOS transistor;
the twenty third PMOS transistor includes a drain connected to node N33, a gate connected to node N39, and a drain connected to N39;
the twenty fourth PMOS transistor includes a drain connected to node N7, a gate connected to N39, and a drain connected to N39;
each of the twenty fifth, twenty sixth, twenty seventh, twenty eighth, and twenty ninth PMOS transistors includes a source, a gate, and a drain;
for the twenty fifth PMOS transistor, the drain is connected to node N41, the gate is connected to node N43, and the source connected to the drain of the twenty sixth PMOS transistor;
for the twenty sixth PMOS transistor, the drain is connected to the source of the twenty fifth PMOS transistor, the gate is connected to N43, and the source connected to the drain of the twenty seventh PMOS transistor;
for the twenty seventh PMOS transistor, the drain is connected to the source of the twenty sixth PMOS transistor, the gate is connected to N43, and the drain connected to the drain of the twenty eighth PMOS transistor;
for the twenty eighth PMOS transistor, the drain is connected to the source of the twenty seventh PMOS transistor, the gate is connected to N43, and the source connected to the drain of the twenty nineth PMOS transistor;
for the twenty nineth PMOS transistor, the drain is connected to the source of the twenty eighth PMOS transistor, the gate is connected to node N43, and the source connected to node N7.
